(19) United States
(12) Patent Application Publication
(10) Pub. No.: US 2005/0197496 A1
(43) Pub. Date: Sep. 8, 2005

(54) Methods of protein fractionation using high performance tangential flow filtration

(75) Inventor: Mark Perreault, Leominster, MA (US)

Correspondence Address: GTC BIOTHERAPEUTICS, INC., 175 CROSSING BOULEVARD, SUITE 410, FRAMINGHAM, MA, 01702, US

(73) Assignee: GTC Biotherapeutics, Inc.

(21) Appl. No.: 11/051,216

(22) Filed: Feb. 4, 2005

Related U.S. Application Data
(60) Provisional application No. 60/550,137, filed on Mar. 4, 2004.

(51) Int. Cl. A61K 38/17; C07K 1/34

(52) U.S. Cl. 530/412

(57) ABSTRACT

Processes and apparati are provided for separating molecules of interest from a mixture containing them which comprises subjecting the mixture to an improved method of high performance tangential flow filtration (HPTFF). The HPTFF of the invention was used to clarify, and process various feedstreams for the removal of a molecule of interest based on both the size and charge of the molecule of interest. According to a preferred embodiment, a transgenic milk feedstream is stabilized and particulate matter such as fat, casein miscelles and bacteria are removed. The method of HPTFF used in the current invention utilizes optimized process parameters that include temperature, trans-membrane pressure, molecular charge, molecular size, cross-flow velocity, and milk concentration. Cleaning and storage procedures were also developed to ensure long membrane life. An aseptic filtration step was also developed to remove any bacteria remaining in a clarified transgenic milk feedstream.

### PRIORITY CLAIM

This application claims priority to U.S. Ser. No. 60/550,137, filed on Mar. 4, 2004, the contents of which are incorporated herein by reference.

### FIELD OF THE INVENTION

The present invention provides an improved method and system of clarifying specific target molecules from contaminants found in an initial feedstream. More specifically the methods of the current invention provide for the processing of a sample solution through an improved method of high performance tangential flow filtration that enhances the clarification, concentration and fractionation of a desired molecule from a given feedstream.

### BACKGROUND OF THE INVENTION

The present invention is directed to an improved method of fractionation of molecules of interest from a given feedstream. It should be noted that the production of large quantities of relatively pure, biologically active molecules is important economically for the manufacture of human and animal pharmaceutical formulations, proteins, enzymes, antibodies and other specialty chemicals. In the production of many polypeptides, antibodies and proteins, various recombinant DNA techniques have become the method of choice since these methods allow the large scale production of such proteins. The various “platforms” that can used for such production includes bacteria, yeast, insect or mammalian cell cultures and transgenic animals. For transgenic animal systems, the preferred animal type is production in mammals, but this platform production method also contemplates the use of avians or even transgenic plants to produce exogenous proteins, antibodies, or fragments or fusions thereof.

Producing recombinant protein involves transfecting host cells with DNA encoding the protein and growing the host cells, transgenic animals or plants under conditions favoring expression of the recombinant protein or other molecule of interest. The prokaryote—E. coli has been a favored host system because it can be made to produce recombinant proteins in high yields. However, numerous U.S. patents on the general expression of DNA encoding proteins exist, for a variety of expression platforms from E. coli to cattle have been developed.

With improvements in the production of exogenous proteins or other molecules of interest from biological systems there has been increasing pressure on industry to develop new techniques to enhance and make more efficient the purification and recovery processes for the biologics and pharmaceuticals so produced. That is, with an increased pipeline of new products, there is substantial interest in devising methods to bring these therapeutics, in commercial volumes, to market quickly. At the same time the industry is facing new challenges in terms of developing novel processes for the recovery of transgenic proteins and antibodies from various bodily fluids including milk, blood and urine. The larger the scale of production the more complex these problems often become. In addition, there are further challenges imposed in terms of meeting product purity and safety, notably in terms of virus safety and residual contaminants, such as DNA and host cell proteins that might be required to be met by the various governmental agencies that oversee the production of biologically useful pharmaceuticals.

Several methods are currently available to separate molecules of biological interest, such as proteins, from mixtures thereof. One important such technique is affinity chromatography, which separates molecules on the basis of specific and selective binding of the desired molecules to an affinity matrix or gel, while the undesirable molecule remains unbound and can then be moved out of the system. Affinity gels typically consist of a ligand-binding moiety immobilized on a gel support. For example, GB 2,178,742 utilizes an affinity chromatography method to purify hemoglobin and its chemically modified derivatives based on the fact that native hemoglobin binds specifically to a specific family of poly-anionic moieties. For capture these moieties are immobilized on the gel itself. In this process, unmodified hemoglobin is retained by the affinity gel, while modified hemoglobin, which cannot bind to the gel because its poly-anion binding site is covalently occupied by the modifying agent, is removed from the system. Affinity chromatography columns are highly specific and thus yield very pure products; however, affinity chromatography is a relatively expensive process and therefore very difficult to put in place for commercial operations.

Typically, genetically engineered biopharmaceuticals are purified from a supernatant containing a variety of diverse host cell contaminants. Reversed-phase high-performance liquid chromatography (RP-HPLC) can be used for protein purification because it can efficiently separate molecular species that are exceptionally similar to one another in terms of structure or weight. Procedures utilizing RP-HPLC have been published for many molecules. McDonald and Bidlingmeyer, “Strategies for Successful Preparative Liquid Chromatography”, PREPARATIVE LIQUID CHROMATOGRAPHY, Brian A. Bidlingmeyer (New York: Elsevier Science Publishing, 1987), vol. 38, pp. 1-104; Lee et al., Preparative HPLC. 8th Biotechnology Symposium, Pt. 1, 593-610 (1988).

Moreover, in another industry that faces some of the same challenges new answers are needed. The dairy industry has been one of the greatest advocates of using membrane systems for fractionation, clarification and purification using the technology since its beginning to concentrate and fractionate whey, as well as treat wastewater, In the 1980s, researchers in the dairy industry began using membranes to concentrate milk for use in the production of non-standardized cheese. In recent years, improved technologies are making membrane-concentrated milk more attractive than ever. At the same time, technological advancements in membrane materials, process engineering and functionality of milk constituents have made membrane separation processes practical and useful at nearly every stage of milk treatment. Though these practices cannot yet be applied to all facets of the dairy industry, their potential is immense.

For example, membrane separation may be particularly attractive to fluid milk processors in the future because it demands little energy and does not destroy any product during treatment. Four basic types of membrane filtration present potential applications for the dairy industry- reverse osmosis (PC), nanofiltration (NF), ultrafiltration (UF) and microfiltration (MF)—each serving a different purpose. Some application processes involve only a single membrane; however, advanced approaches are using two or more membrane processes in a given application. However, these processes, though useful, are still limiting with regard to some aspects of the dairy industry, food preparation industry and biopharmaceutical production in transgenic animals.

In both the biotech industry and in the dairy industry ultrafiltration has traditionally been used for size-based separation of protein mixtures where the ratio of the protein molecular masses have to be at least around 10 to 1. This has been a limiting factor in many industrial applications throughout industry and in particular in the recovery of biopharmaceuticals in the milk of transgenic mammals. Significant research has taken place in the optimization of ultrafiltration systems by altering the physiochemical conditions (i.e. pH and ionic strength) to achieve higher selectivities (Van Reis et al. (1997)). According to the methods of the current invention improvements have been made to optimize conditions more in the direction of pH and ionic strength to make possible the development of high-performance tangential flow filtration (HPTFF) in various feedstreams including milk.

HPTFF exploits multiple phenomena to maximize separation performance. These include the manipulation of solution pH and ionic strength to maximize differences in solute effective volumes as well as the use of membranes with controlled pore size.

As mentioned, current industrial and biopharmaceutical processes often use ion-exchange chromatography, UF and size exclusion chromatography (SEC) in three separate steps for purification, concentration and buffer exchange. However, even in conjunction with one another, these processes are limited in terms of what they can separate. Even ultrafiltration (UF) is generally limited to separation of solutes that differ by at least tenfold in size. In addition, molecular species that are similar in charge can also be very difficult to separate. HPTFF is a two-dimensional purification method that exploits differences in both size and charge characteristics of biomolecules, It is hence possible to separate biomolecules with the same molecular weight. It is even possible to retain one biomolecule while passing a larger molecular weight species through the membrane.

Molecules that differ less than threefold in size can he separated through the use of highly selective charged membranes and careful optimization of buffer and fluid dynamics. Knowledge of the isoelectric point (pI) of the desired molecule of interest is the main factor in HPTFF. This will then dictate membrane set-up and the intrinsic charge profile of the membrane, pore size, and flow characteristics. Moreover, HPTFF makes it possible to perform all of these steps in a single-unit operation, thereby reducing production costs. In addition, HPTFF uses the same linear scale-up principles already established for UF. HPTFF is also assisted by optimizing the trans-membrane pressure.

Depending on membrane type, it can be classified as microfiltration or ultrafiltration. Microfiltration membranes, with a pore size between 0.1 and 10 μm, are typically used for clarification, sterilization, removal of microparticulates, or for cell harvests. Ultrafiltration membranes, with much smaller pore sizes between 0.001 and 0.1 μm, are used for separating out and concentrating dissolved molecules (protein, peptides, nucleic acids, carbohydrates, and other biomolecules), for exchange buffers, and for gross fractionation.

However, limitations exist on the degree of protein purification achievable in ultrafiltration. These limits are due mainly to the phenomena of concentration polarization, fouling, and the wide distribution in the pore size of most membranes. Therefore, solute discrimination is often poor. See, e.g., Porter, ed., HANDBOOK OF INDUSTRIAL MEMBRANE TECHNOLOGY (Noyes Publications, Park Ridge, N.J., 1990), pp. 164-173. A polarized layer of solutes acts as an additional filter and essentially acts in series with the original ultra-filter. This action provides significant resistance to the filtration of a given solvent. The degree of polarization increases with increasing concentration of retained solute in the feed, and can lead to a number of seemingly anomalous or unpredictable effects in real systems. For example, under highly polarized conditions, filtration rates may increase only slightly with increasing pressure, in contrast to unpolarized conditions, where filtration rates are usually linear with pressure. Use of a more open, higher-flux membrane may not increase the filtration rate, because the polarized layer is providing the limiting resistance to filtration. The situation is further complicated by interactions between retained and eluted solutes. A result of concentration polarization and fouling processes is the inability to make effective use of the macromolecular fractionation capabilities of ultrafiltration membranes for the large-scale resolution of macromolecular mixtures such as blood plasma proteins. See Michaels, “Fifteen Years of Ultrafiltration: Problems and Future Promises of an Adolescent Technology”, in ULTRAFILTRATION MEMBRANES AND APPLICATIONS, POLYMER SCIENCE AND TECHNOLOGY, 13 (Plenum Press, N.Y., 1979, Anthony R. Cooper, ed.,), pp. 1-19.

TFF and HPTFF can be further subdivided into categories based on the size of components being separated. For protein processing, these can range from the size of intact cells to buffer salts. Table 1 below details typical components that would be retained by a membrane and that would pass through a membrane for each of the subdivisions. In addition, it shows the range of membrane pore size ratings or nominal molecular weight limits (NMWL) that generally fall into each category.

TABLE 1
Subdivisions of Tangential Flow Filtration Processes.

| | | Virus | High-Perfomance | Ultrafiltration | Nanofiltration/ |
|---|---|---|---|---|---|
| | Mkrofiltration | Filtration | Filtration | TFF | Reverse Ognosis |
| Components retained | Intact cells | Viruses | Proteins | Proteins | Antibiotics |
| by membrane | Cell debris | | | | Sugars |
| | | | | | Salts |

membrane membrane membrane membrane membrane membrane membrane membrane membrane
membrane membrane membrane membrane membrane

| | | | | | |
|---|---|---|---|---|---|
| Components passed | Collodial material | Proteins | Proteins | Small Peptides | (Salts) |
| through membrane | Viruses | Salts | Salts | Salts | water |
| | Proteins | | | | |
| | Salts | | | | |
| Approximate membrane | 0.05 μm-1 μm | 100 KD-0.05 μm | 10 KD-300 KD | 1 KD-1000 KD | <1 KD |
| cutoff range | | | | | |

The use of tangential flow filtration for the separation of materials is known. Marinaccio et al., U.S. Pat. No. 4,888,115 discloses the process (termed “cross-flow”) for use in the separation of biological liquids such as blood components for plasmapheresis. In this process, blood is passed tangentially to (i.e., across) an organic polymeric microporous filter membrane, and particulate matter is removed. In another example of current art, tangential flow filtration has been disclosed for the filtration of beer solutions (Shackleton, EP 0,208,450, published Jan. 14, 1987) specifically for the removal of particulates such as yeast cells and other suspended solids. Kothe et al., (U.S. Pat. No. 4,644,056, issued Feb. 17, 1987) disclose the use of this process in the purification of immunoglobulins from milk or colostrum, and Castino (U.S. Pat. No. 4,420,398, issued Dec. 13, 1983) describes its use in the separation of antiviral substances such as interferons from broths containing these substances as well as viral particles and the remains of cell cultures from which they are derived.

TFF units have been employed in the separation of bacterial enzymes from cell debris (Quirk et al., 1984, ENZYME MICROB. TECHNOL., 6(5):201). Using this technique, Quirk et al. were able to isolate enzyme in higher yields and in less time than using the conventional technique of centrifugation. The use of tangential flow filtration for several applications in the pharmaceutical field has been reviewed by Genovesi (1983, J. PARENTER. Aci. TECHNOL., 37(3):81), including the filtration of sterile water for injection, clarification of a solvent system, and filtration of enzymes from broths and bacterial cultures.

However, the precise control of particle size needed for commercial applications of the technology is difficult and generally has not been successful. In the present invention the use of tangential flow filtration has been improved to separate particles according to size and charge in a commercially efficient and important process. The resulting HPTFF system is employed through the current invention to improve clarification and fractionation efforts even from the levels achieved by TFF. The use of filters of selected sizes, and further, the sequential use or serial attachment of filters of different sizes (i.e., a filtering system) is disclosed for the separation of particles to obtain particles of a specifically desired size range.

One such molecule of interest that can be purified from a cell culture broth or a transgenic milk feedstream is human recombinant alphafetoprotein. Other molecules of interest include without limitation, human albumin, antibodies, Fc fragments of antibodies and fusion molecules wherein a human albumin or alpha-fetoprotein protein fragment acts as the carrier molecule.

The methods of the current invention also provide precise combinations of filters and conditions that allow the optimization of the yield of molecules of interest from a given feedstream. In these methods important the process parameters such as pH and temperature are precisely manipulated.

The biologics industry is becoming increasingly concerned with product safety and purity as well as cost of goods. The use of HPTFF, according to the current invention, is a rapid and more efficient method for biomolecule separation. It can be applied to a wide range of biological fields such as immunology, protein chemistry, molecular biology, biochemistry, and microbiology.

### BRIEF DESCRIPTION OF THE DRAWINGS

FIG. 1 Shows a process flow diagram for flow of material from feedstream through HPTFF to fill and finish.

FIG. 2A Shows the process and equipment set-up for microfiltration.

FIG. 2B Shows the process and equipment set-up for TFF.

FIG. 3 Shows fluid flowpaths through different TFF and HPTFF modules

FIG. 4 Shows a filtration process flow diagram.

FIG. 5 Shows the transgenics development process from a DNA construct to the production of clarified milk containing a recombinant protein of interest.

FIG. 6 Shows a process equipment schematic for the methods of the current invention.

FIG. 7 Shows open and turbulence-promoted feed channels in HPTFF module types

FIG. 8 Shows the HPTFF system of the Invention.

### SUMMARY OF THE INVENTION

Briefly stated, the objective of the current invention is to use HPTFF techniques to achieve more efficient protein fractionation. That is, to improve the separation of the protein of interest from contaminating proteins using HPTFF. One protein of interest used as an example, recombinant human alphafetoprotein is a protein of approximately 66 KD in molecular weight and has a structure similar to that of albumin. The goal of the methods of the current invention are to retain the target protein (rhAFP) and pass the major contaminating milk proteins in the most efficient manner possible. According to the current invention, contaminating milk proteins include IgG, Lactoferrin, albumin, casein, lactoglobulin, and lactalbumin. According to a preferred embodiment of the current invention all but the recombinant human alphafetoprotein and goat albumin are effectively reduced in concentration using a 100 KD tangential flow membrane method. By reducing the concentration of contaminating proteins the recombinant human alphafetoprotein retained is enhanced in both purity and stability and is able to be further purified using conventional chromatography more efficiently.

The methods of the current invention use rhAFP as an exemplar but can be used for other proteins of interest. The recombinant human alphafetoprotein is retained using a 100 KD MWCO membrane and the other proteins then freely pass through the membrane as the solution is continuously diafiltered with 20 mM Phosphate buffer.

The initial recombinant human alphafetoprotein protein purity in clarified milk was approximately 5-7 percent in purity by SDS page. Following the protein fractionation the relative purity rises to approximately 30 percent with a yield of 85%. This initial fractionation of the current invention improves the downstream process efficiency as the protein arrives in a semi-purified state accelerating the processing of human therapeutic proteins, protein fragments, or antibodies from a variety of feedstreams, preferably from transgenic mammalian milk.

Therefore, in a preferred embodiment of the current invention the filtration technology developed and provided herein provides a process to clarify, concentrate and fractionate the desired recombinant protein or other molecule of interest from the native components of milk or contaminants thereof. The resulting clarified bulk intermediate is a suitable feed material for traditional purification techniques such as chromatography which are used down stream from the HPTFF process to bring the product to it's final formulation and purity.

A preferred procotol of the current invention employs three filtration unit operations that clarify, concentrate, and fractionate the product from a given transgenic milk volume containing a molecule of interest. The clarification step removes larger particulate matter, such as fat globules and casein micelles from the product. The concentration and fractionation steps thereafter remove most small molecules, including lactose, minerals and water, to increase the purity and reduce the volume of the resulting product composition. The product of the HPTFF process is tailor concentrated to a level suitable for optimal down stream purification and overall product stability. This concentrated product is then aseptically filtered to assure minimal bioburden and enhance stability of the product for extended periods of time. The bulk product will realize a purity between 65% and 85% and may contain components such as albumin, whey proteins (β Lactoglobulin, α Lactalbumin, and BSA), and low levels of residual fat and casein. This partially purified product is an ideal starting feed material for conventional down stream chromatographic techniques.

Typical of the products that the current invention can be used to process are transgenically produced proteins of interest, including without limitation: alphafetoprotein, IgG1 antibodies, fusion proteins (ex: erythropoietin—human albumin fusion—“HEAP” or Human Albumin—Erythropoietin; Beta—Interferon—Alphafetoprotein fusion), antithrombin III, alpha-1-antitrypsin, IgG4, IgM, IgA, Fc portions, fusion molecules containing a peptide or polypeptide joined to a immunoglobulin fragment. Other proteins that can be processed by the current invention include recombinant proteins, exogenous hormones, endogenous proteins or biologically inactive proteins that can be later processed to restore biological function. Included among these processes, without limitation, are human growth hormone, recombinant human albumin, decorin, human alpha fetoprotein urokinase, tPA and prolactin.

Moreover, according to the current invention the alterations in salt (NaCl) concentration and the two diafiltration steps differ from the prior art and serve to enhance the purity available according to those using the methods of the current invention.

It is an object of the present invention to provide more efficient high performance tangential-flow filtration processes for separating species such as particles and molecules by size, which processes are selective for the species of interest, resulting in higher-fold purification thereof.

It is another object to provide improved filtration processes, including ultrafiltration processes, for separating biological macromolecules such as proteins which processes minimize concentration polarization and do not increase flux.

It is another object to provide a filtration process that can separate by size species that are less than ten-fold different in size and do not require dilution of the mixture prior to filtration.

These and other objects will become apparent to those skilled in the art. Other features and advantages of this invention will become apparent in the following detailed description of preferred embodiments of this invention, taken with reference to the accompanying drawings.

### DESCRIPTION OF THE PREFERRED EMBODIMENT

The following abbreviations have designated meanings in the specification:

Abbreviation Key:

BSA Bovine Serum Albumin
CHO Chinese Hamster Ovary cells
CV Crossflow Velocity
DFF Direct Flow Filtration
DV Diafiltration Volume
IEF Isoelectric Focusing
GMH Mass Flux (grams/m²/hour)—also J_{M}
LMH Liquid Flux (liters/m²/hour)—also J_{L}
LPM Liters Per Minute
M Molar
MF Microfiltration
NMWCO Nominal Molecular Weight Cut Off
NWP Normalized Water Permeability
PES Poly(ether)-sulfone
pH A term used to describe the hydrogen-ion activity of a chemical or compound according to well-known scientific parameters.
PPM Parts Per Million
SDS-PAGE SDS (sodium dodecyl sufate) Poly-Acrylamide Gel electrophoresis
SEC Size Exclusion Chromatography
TFF Tangential Flow Filtration
PEG Polyethylene glycol
TMP Transmembrane Pressure
UF Ultrafiltration
   Explanation of Terms:
   Clarification
   The removal of particulate matter from a solution so that the solution is able to pass through a 0.2 μm membrane.
   Colloids
   Refers to large molecules that do not pass readily across capillary walls. These compounds exert an oncotic (i.e., they attract fluid) load and are usually administered to restore intravascular volume and improve tissue perfusion.
   Concentration
   The removal of water and small molecules with a membrane such that the ratio of retained molecules to small molecules increases.
   Concentration Polarization
   The accumulation of the retained molecules (gel layer) on the surface of the membrane caused by a combination of factors: transmembrane pressure, crossflow velocity, sample viscosity, and solute concentration.
   Crossflow Velocity
   Velocity of the fluid across the top of the membrane surface. CF=Pi−Po where Pi is pressure at the inlet and Po is pressure at the outlet and is related to the retentate flow rate.
   Diafiltration
   The fractionation process of washing smaller molecules through a membrane, leaving the larger molecule of interest in the retentate. It is a convenient and efficient technique for removing or exchanging salts, removing detergents, separating free from bound molecules, removing low molecular weight materials, or rapidly changing the ionic or pH environment. The process typically employs a a microfiltration membrane that is employed to remove a product of interest from a slurry while maintaining the slurry concentration as a constant.
   Feedstream
   The raw material or raw solution provided for a process or method and containing a protein of interest and which may also contain various contaminants including microorganisms, viruses and cell fragments. A preferred feedstream of the current invention is transgenic milk containing a exogenous protein of interest.
   Filtrate Flux (J)
   The rate at which a portion of the sample has passed through the membrane.
   Flow Velocity (V)
   The speed at which the fluid passes the surface of the membrane is considered the fluid flow velocity. Product flux will be measured as flow velocity is varied. The relationship between the two variables will allow us to determine an optimal operational window for the flow.
   Fractionation
   The preferential separation of molecules based on a physical or chemical moiety.
   Gel Layer
   The microscopically thin layer of molecules that can form on the top of a membrane. It can affect retention of molecules by clogging the membrane surface and thereby reduce the filtrate flow.
   High Performance Tangential Flow Filtration
   HPTFF is a high resolution process where protein-protein separations can be carried out on the basis of both size and charge, resulting in product yields and purification factors similar to chromatography. Membrane NMWLs used for HPTFF are in the range of 10 kD to 300 kD.
   Membrane Pore Size Rating (MPSR)
   A membrane pore size rating, typically given as a micron value, indicates that particles larger than the rating will be retained by the membrane.
   Nominal Molecular Weight Cut Off (NMWCO)
   The size (kilodaltons) designation for the ultrafiltration membranes. The NMWCO is defined as the molecular weight of the globular protein that is 90% retained by the membrane.
   Nominal Molecular Weight Limits (NMWL)
   A membrane rating system that indicates that most dissolved macromolecules with molecular weights higher than the NMWL and some with molecular weights lower than the NMWL will be retained by the membrane in question.
   Normalized Water Permeability (NWP)
   The water filtrate flow rate established at a specific recirculation rate during TFF device initial cleaning. This value is used to calculate membrane recovery.
   Molecule of Interest
   Particles or other species of molecule that are to be separated from a solution or suspension in a fluid, e.g., a liquid. The particles or molecules of interest are separated from the fluid and, in most instances, from other particles or molecules in the fluid. The size of the molecule of interest to be separated will determine the pore size of the membrane to be utilized. Preferably, the molecules of interest are of biological or biochemical origin or produced by transgenic or in vitro processes and include proteins, peptides, polypeptides, antibodies or antibody fragments. Examples of preferred feedstream origins include mammalian milk, mammalian cell culture and microorganism cell culture such as bacteria, fungi, and yeast. It should also be noted that species to be filtered out include non-desirable polypeptides, proteins, cellular components, DNA, colloids, mycoplasm, endotoxins, viruses, carbohydrates, and other molecules of biological interest, whether glycosylated or not.
   Tangential Flow Filtration
   A process in which the fluid mixture containing the components to be separated by filtration is re-circulated at high velocities tangential to the plane of the membrane to increase the mass-transfer coefficient for back diffusion. In such filtrations a pressure differential is applied along the length of the membrane to cause the fluid and filterable solutes to flow through the filter. This filtration is suitably conducted as a batch process as well as a continuous-flow process. For example, the solution may be passed repeatedly over the membrane while that fluid which passes through the filter is continually drawn off into a separate unit or the solution is passed once over the membrane and the fluid passing through the filter is continually processed downstream.
   Transmembrane Pressure
   The pressure differential gradient that is applied along the length of a filtration membrane to cause fluid and filterable solutes to flow through the filter. In tangential flow systems, highest TMP's are at the inlet (beginning of flow channel) and lowest at the outlet (end of the flow channel). TMP is calculated as an average pressure of the inlet, outlet, and filtrate ports.
   Recovery
   The amount of a molecule of interest that can be retrieved after processing. Usually expressed as a percentage of starting material or yield.
   Retentate
   The portion of the sample that does not pass through the membrane, also known as the concentrate. Retentate is being re-circulated during the TFF.
   Basics of Tangential Flow Filtration

There are two important variables involved in all tangential flow devices: the transmembrane pressure (TMP) and the crossflow velocity (CF). The transmembrane pressure (TMP) is the force that actually pushes molecules through the pores of the filter. The crossflow velocity is the flow rate of the solution across the membrane. It provides the force that sweeps away larger molecules that can clog the membrane thereby reducing the effectiveness of the process. In practice a fluid feedstream is pumped from the sample feed container source across the membrane surface (crossflow) in the filter and back into the sample feed container as the retentate. Backpressure applied to the retentate tube by a clamp creates a transmembrane pressure which drives molecules smaller than the membrane pores through the filter and into the filtrate (or permeate) fraction. The crossflow sweeps larger molecules, which are retained on the surface of the membrane, back to the feed as retentate. The primary objective for the successful implementation of a TFF protocol is to optimize the TMP and CF so that the largest volume of sample can be filtered without creating a membrane-clogging gel. A TMP is “substantially constant” if the TMP does not increase or decrease along the length of the membrane generally by more than about 10 psi of the average TMP, and preferably by more than about 5 psi. As to the level of the TMP throughout the filtration, the TMP is held constant or is lowered during the concentration step to retain selectivity at higher concentrations. Thus, “substantially constant TMP” refers to TMP versus membrane length, not versus filtration time.

Overview

According to the preferred embodiment of the current invention, the transgenic (“TG”) milk is initially clarified using microfiltration to remove fat globules and casein micelles. The permeate from the microfiltration is recirculated through a 30 kD TFF cassette system where the milk proteins are retained; salt and sugars are passed through the membrane and recycled to the microfiltration retentate as diafiltration buffer. The recombinant human alphafetoprotein product resides in the clarified milk that is retained by the 30 kd membrane. The recombinant human alphafetoprotein is now in a solution with a complex mixture of milk proteins, some in a great abundance. The 100 kD protein fractionation step is designed to reduce the amount of contaminating milk proteins and prepare the recombinant human alphafetoprotein for purification using chromatography.

Before the 100 kD fractionation can be carried out the clarified milk containing the protein of interest must be buffer exchanged to remove the salts found in the milk. Therefore once the clarification is complete the protein of interest (e.x.: recombinant human alphafetoprotein) can then diafiltered 5 times using the same 30 kD TFF cassette with 20 mM Phosphate Buffer at pH 6.5.This initial diafiltration is necessary to reduce the salt concentration of the clarified milk. By reducing the salt concentration the hydrodynamic radius of the recombinant human alphafetoprotein increases and allows the protein to be easily retained by a 100 kD MWCO, HPTFF membrane. The other milk proteins (with the exception of goat albumin) are not affected in the same manner as the recombinant human alphafetoprotein. They will therefore pass freely through the 100 kd membrane and be removed and discarded as waste.

The objectives of the 100 KD protein fraction are to remove unwanted milk proteins, lipids, and low molecular weight contaminants prior to chromatography. By effectively removing the contaminants using a diafiltration, less of a burden is put on to the remaining chromatographic steps of the process.

Milk as a Feedstream

According to a preferred embodiment of the current invention, the HPTFF process employs three filtration unit operations that clarify, concentrate, and fractionate the product from a milk feedstream. This milk may be the product of a transgenic mammal containing a biopharmaceutical or other molecule of interest. In a preferred embodiment the system is designed such that it is highly selective for the molecule of interest. The clarification step removes larger particulate matter, such as fat globules and casein micelles from the milk feedstream. The concentration/fractionation steps remove most small molecules, including lactose, minerals and water, to increased purity and reduce volume of the product. The product of the TFF process is thereafter concentrated to a level suitable for optimal downstream purification and overall product stability. This concentrated product, containing the molecules of interest, is then aseptically filtered to assure minimal bio-burden (i.e., endotoxin) and enhance the stability of the molecules of interest for extended periods of time. According to a preferred embodiment of the current invention, the bulk product will realize a purity between 65% and 85% and may contain components such as goat antibodies (from transgenic goats), whey proteins (β Lactoglobulin, α Lactalbumin, and BSA), as well as low levels of residual fat and casein. This partially purified product is an ideal starting feed material for conventional downstream chromatographic techniques to further select and isolate the molecules of interest which could include, without limitation, a recombinant protein produced in the milk, an immunoglobulin produced in the milk, or a fusion protein.

### Step # 1 (Clarification)

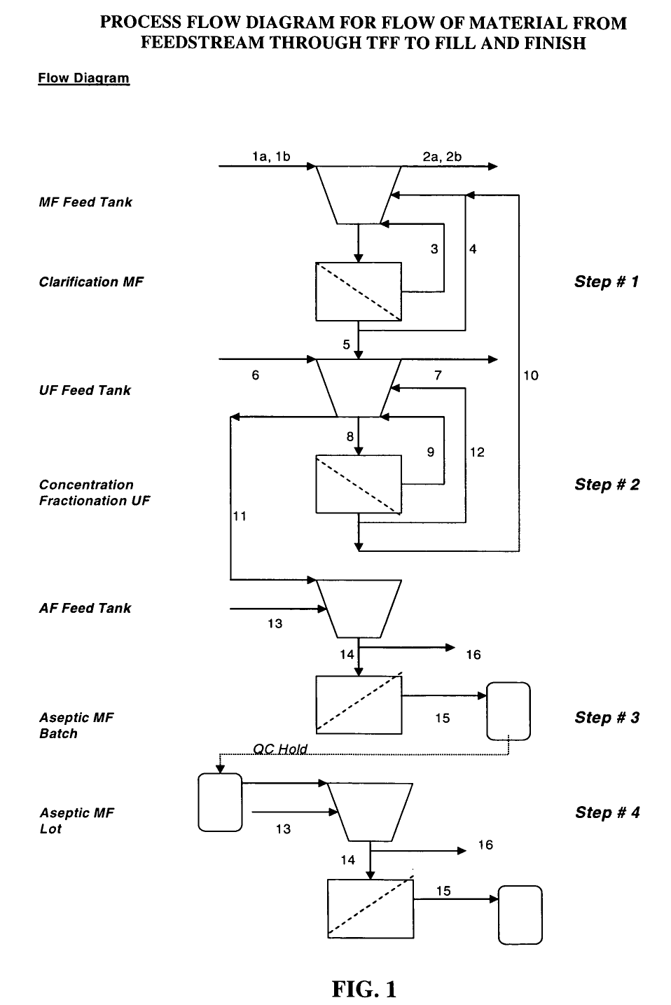

Turning to FIG. 1, transgenic mammal milk, preferably of caprine or bovine origin, is clarified utilizing batch-wise microfiltration. The milk is placed into a feed tank and pumped in a loop to concentrate the milk retentate two fold (see flow diagram in FIG. 1). Once concentrated the milk retentate is then diafiltered allowing the product and small molecular weight proteins, sugars, and minerals to pass through an appropriately sized membrane. According to the current invention, this operation is currently designed to take 2 to 3 hours and is will process 1000 liters of milk per day. The techniques and methods of the current invention can be scaled up and the overall volume of product that can be produced is dependent upon the commercial and/or therapeutic needs for a specific molecule of interest.

### Step # 2 (Concentration/Fractionation)

Again referring to FIG. 1., the clarified permeate from the first step is concentrated and fractionated using ultrafiltration (“UF”). The clarified permeate flows into the UF feed tank and is pumped in a loop to concentrated the product two-fold. Once the concentration step is initiated the permeate from the UF is placed into the milk retentate in the clarification feed tank in the first step. The first and second step are sized and timed to be processed simultaneously. The permeate from the UF contains small molecular weight proteins, sugars, and minerals that pass through the membrane. Once 95% of the product is accumulated in the retentate of the UF, the clarification is stopped and a concentration/diafiltration of the UF material is begun. The product is concentrated 5 to 10 fold the initial milk volume and buffer is added to the UF feed tank. This washes away the majority of the small molecular weight proteins, sugars, and minerals. This operation is currently designed to take 2.5 to 3.5 hours and can process upto 500 liters of clarified permeate per day. As above, the techniques and methods of the current invention can be scaled up and the overall volume of product that can be produced is dependent via this concentration/fractionation process is dependent upon the commercial and/or therapeutic needs for a specific molecule of interest.

### Step # 3 (Aseptic Filtration)

According to FIG. 1., and according to the current invention, the clarified bulk concentrate is then aseptically microfiltered. The resulting 50 to 100 liters of UF retentate is placed into a feed tank where it is pumped through a dead-end absolute 0.2 μm MF filtering system in order to remove the majority of the bio-burden and enhance stability of the product for extended periods of time. The product is pumped through the filtering system of the invention and may then be directly filled into a final packaging configuration. Under conditions for processing a molecule of interest in a GMP facilities meeting clean room specifications (e.g., class 100 conditions) This operation is currently designed to take 0.5 to 1 hour and will process upto 100 liters of clarified bulk intermediate per day. As above, the techniques and methods of the current invention can be scaled up and the overall volume of product that can be produced is dependent via this concentration/fractionation process is dependent upon the commercial and/or therapeutic needs for a specific molecule of interest.

### EXAMPLE 1

### Milk as a Feedstream for the Clarification of a Molecule of Interest

The data below provides an application of the current invention that provides a membrane-based process to clarify, concentrate, and fractionate transgenically produced a protein of interest (e.x..: human recombinant alphafetoprotein) from a raw milk feedstream. According to this example of the invention the transgenic mammal providing the milk for processing was a goat but other mammals may also be used including cattle, rabbits, mice as well sheep and pigs.

The starting material for the protein fractionation had already been clarified using microfiltration and then set aside for the initial membrane optimization studies. A set of experiments were designed to evaluate the effect of each of these parameters and pinpoint the optimal conditions for the separation of recombinant human alphafetoprotein from the contaminating milk proteins. One variable was changed at a time initially until each one was optimized and showed the proper set of conditions for the fractionation. The following ranges of parameters were chosen for the fractionation experiments:

| | | |
|---|---|---|
| I. | Membrane Molecular Weight | 50-100 kD |
| | Cutoff (MWCO) | |
| II. | Transmembrane Pressure (TMP) | 5-30 psi |
| III. | Clarified Milk pH and Ionic Strength | 0M-1.0M NaCl |
| | (20 mM Phos. pH 6.5) | |
| IV. | Clarified Milk Concentration Factor (CFac) | 1X-4X |
| V. | Number of Diafiltration Volumes | 12-20 DV's |
| VI. | Clarified Milk Lot | (see materials) |
| VII. | Membrane Recovery | (see section VII) |

The TFF system was sanitized using 0.1M NaOH, flushed with USP water, and equilibrated using 20 mM Sodium Phosphate Buffer at pH 6.5. The initial water permeability rates were measured and recorded. Four liters of clarified milk was initially concentrated by a factor times and reduced to a volume of one liter. The concentrated clarified milk was then diafiltered using 20 mM Sodium Phosphate Buffer at pH 6.5. Rather than diafiltering the milk a fixed number of diafiltration volumes, it was instead diafiltered to an O.D. of 4.0 at 280 nm. This absorbance roughly correlates to 6 g/l of total protein. This concentration of total protein was chosen due to restraints put on the process by the following chromatographic step. Once the diafiltration was complete the system was drained and flushed with one liter of Phosphate Buffer that was then combined with the final retentate. The fractionated recombinant human alphafetoprotein was then sterile filtered using an 0.2 um capsule filter.

The fractionated recombinant human alphafetoprotein was then analyzed for total protein, AFP concentration, and contaminating proteins using RPC. Additionally an SDS gel was run to further evaluate the remaining contaminating proteins.

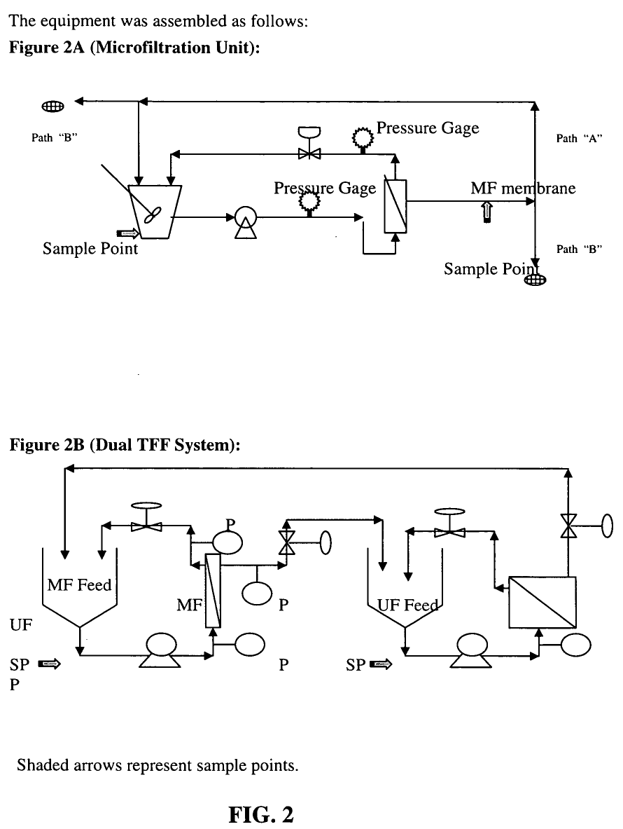

The HPTFF system of the invention consists of a Pall Centramate four gauge system with two peristaltic pumps. The first pump was used to re-circulate the retentate and the second to re-ciurculate the permeate. This pumping scheme is known as Co-Current flow, see FIGS. 1 and 2. It is most commonly used to balance the TMP along the entire path length of the membrane, ensuring a more uniform fractionation.

[image]

[image]

[image]

II. Transmembrane Pressure (TMP)

The development of the protein fractionation step included process optimization using the 100 KD HPTFF membrane in a recirculating mode. Initially the process conditions were characterized by comparing TMP vs. Flux (see below). The starting material was clarified milk diafiltered with 20 mM Phosphate buffer at pH 6.5. The buffer conditions suited this fractionation as low salt concentration increases the retention of the rhAFP molecule. The optimization shows our optimal TMP should be between 12 and 20 psi, which is beyond the membrane controlled region and into the transition zone where the membrane is layered controlled. The cross flow velocity was not further optimized, but rather held at 0.6 L/min/ft2, recommended by the manufacturer.

[image]

Once the optimization was complete a set of operating parameters could be established for the HPTFF fractionation. These operating parameters established for the protein fractionation are critical for maintaining a reproducible process. Critical parameters that require monitoring include transmembrane pressure, cross flow velocity, and buffer conditions.

[image]

[image]

IV. Clarified Milk Concentration Factor (CFac)

The protein fractionation consisted of an initial 4× concentration to reduce the volume and conserve the amount of buffer required for the diafiltration. The concentration step was not initially used during the development process, but later proved to be necessary as the volume of buffer required for diafiltration at 1× was excessive. The concentrated clarified milk was then diafiltered between 10 and 20 volumes.

### Fractionation at 1×

Clarified milk was initially diafiltered 12 times using 20 mM Sodium Phosphate Buffer at pH 6.5. Once the diafiltration begins the flux begins to rise as the contaminating proteins are removed from the retentate.

[image]

[image]

TABLE 2

Method

| | RPC | RPC |
|---|---|---|
| | Conc. rhAFP | Conc. Total Protein |
| Sample | mg/ml | mg/ml |
| MP 082003 | 0.61 | 1.8 |
| MP 090803 Final 1X | 0.57 | 1.4 |
| MP 090303 Final 1X | 0.56 | 1.4 |

Run#1: 93% Yield

Run#2: 92% Yield

TABLE 3
HPTFF Operating Parameters

| | |
|---|---|
| TMP | 12 psi |
| Cross Flow | 0.6 L/min/ft2 |
| Retentate Pressure Drop | 15-20 psi |
| Permeate Pressure Drop | 15-20 psi - Co-Current Flow |
| Typical Flux | 80-100 LMH |

### Fractionation at 4×

Clarified milk was initially concentrated by a factor times and reduced in volume to 25% of the starting volume. The initial drop in flux during the concentration can be seen at the DV 0 point of the graph. The concentrated clarified milk was then diafiltered 20 times using 20 mM Sodium Phosphate Buffer at pH 6.5. Once the diafiltration begins the flux begins to rise as the contaminating proteins are removed from the retentate. The final 4× concentrate is then flushed from the TFF system with an equal volume of buffer reducing the final concentration to 2 times the starting volume. V,9-10/2

TABLE 4
Run#1: 82% Yield
Run#2: 92% Yield

Method

| | RPC | RPC |
|---|---|---|
| | Conc. rhAFP | Conc. Total Protein |
| Sample | mg/ml | mg/ml |
| MP 082003 | 0.61 | 1.8 |
| MP 090403 Final 2X | 1.00 | 2.5 |
| MP 090903 Final 2X | 1.12 | 2.80 |

TABLE 5
TFF Operating Parameters

| | |
|---|---|
| TMP | 12 psi |
| Cross Flow | 0.6 L/min/ft2 |
| Retentate Pressure Drop | 15-20 psi |
| Permeate Pressure Drop | 15-20 psi - Co-Current Flow |
| Typical Flux | 80-100 LMH |

V. Number of Diafiltration Volumes

One alternative to fractionating the clarified milk with a fixed number of diafiltration volumes is instead to diafilter until the retentate falls to an O.D. at 280 nm. The exact point at which to stop the diafiltration was determined by the absorbance of the 5 retentate at 280 nm. The target absorbance was 4.0 AU at 4× concentration. This absorbance of 4.0 at 280 nm roughly corresponds to 6 mg/ml of total protein by RPC. This allows the process to consistently produce a fractionated product at the same total protein concentration regardless of the starting concentration. The target set for the final total protein concentration is 2.8-3.2 mg/ml once diluted 1:1 with 20 mM Phosphate buffer flush. v,11/2

The graph immediately shows the trend of diafiltration volume vs. flux and diafiltration volume vs. absorbance. The absorbance follows a somewhat exponential decay as the diafiltration progresses. Once the majority of passing proteins have been removed the retaining proteins constitute the final fractionated product. This final product typically contains mostly the recombinant human alphafetoprotein protein, Albumin, and casein

TABLE 6
Percent Yield (by RPC)

| | Conc. hAFP | Total Protein |
|---|---|---|
| Sample | in mg/ml | in mg/ml |
| 201-TR-0001A Sample #3 | 0.61 | N/A |
| 201-TR-0001A Sample #7 | 0.94 | 2.98 |

Two dilutions of each sample were prepared and tested in duplicates. Concentrations reported are average of four injections.

The RPC analysis shows two important pieces of data, recombinant human alphafetoprotein concentration and total protein concentration. The table above shows the initial recombinant human alphafetoprotein concentration as 0.61 mg/ml before the fractionation begins. Following the fractionation the final concentration is 0.94 mg/ml at a concentration factor of two times. The final yield may be calculated in the following manner assuming a target 2× concentration factor.
% Yield=((Final Concentration/Concentration Factor)/Initial Concentration)×100% Yield=((0.94 mg/ml/2)/0.61 mg/ml)×100=78% Yield

*Note: This yield is only an approximation as the Concentration Factor used is only a target value

TABLE 7
Total Protein (by RPC)

| | Conc. hAFP | Total Protein |
|---|---|---|
| Sample | in mg/ml | in mg/ml |
| 201-TR-0001A Sample #3 | 0.61 | N/A |
| 201-TR-0001A Sample #7 | 0.94 | 2.98 |

Two dilutions of each sample were prepared and tested in duplicates. Concentrations reported are average of four injections.

The final recombinant human alphafetoprotein sample is tested for its total protein concentration using rpc. This is information that will be later used for column loading. The target total protein concentration for the final fractionated recombinant human alphafetoprotein was initially targeted at 6 mg/ml at 4 × concentration. The fractionated recombinant human alphafetoprotein was then diluted with an equal volume of flush buffer, bringing the final target total protein concentration to 3.0 mg/ml.

Protein Purity (by RPC & SDS PAGE)

The fractionated recombinant human alphafetoprotein was then analyzed for contaminating proteins using RPC. Additionally an SDS PAGE was run to further evaluate the remaining contaminating proteins.

[image]

Protein Purity (by RPC & SDS PAGE)

The RPC chromatograms above help to demonstrate the protein fractionation performed by the 100 KD tangential flow membrane. The first RPC chromatogram shows a 1.0 mg/ml recombinant human alphafetoprotein purified reference with an elution time of 4.2 minutes. The second RPC chromatogram shows the clarified starting milk prior to the protein fractionation. The third RPC chromatogram shows the fractionated recombinant human alphafetoprotein (peak number 6) and the remaining contaminating milk proteins. Peaks 1 & 2 are unidentified milk proteins, peaks 3 & 5 are Casein, peak number 4 is caprine serum albumin, and peak 7 a high molecular weight milk protein. The difference between the second and third chromatogram show the relative amount of contaminating proteins removed by the protein fractionation step.

[image]

[image]

[image]

[image]

VI. Membrane Recovery

Normalized water permeability (NWP) is a measure of membrane performance recovery from run to run. Without a means to measure the effectiveness of the cleaning of a membrane performance could be lost over the life of the membrane. The NWP of a new membrane is measured and serves as a reference point for all further NWP data recorded. The membrane is typically considered “clean” when greater than 90% of the original NWP has been recovered. The following graph shows the trend of one Pall OS 100C12 cassette used during development. v,17/2

The most effective way to show the effects of using a cassette that has not been cleaned properly is to actually run the process with a fouled membrane. The following graph shows the process flux of three separate runs where the membrane's NWP had not been recovered beyond 60% of the original NWP. Run numbers 081403A, 081403B, 081503A, and 081503B all show a process flux in the range of 50-70 LMH. Once these four runs were complete the membrane was cleaned thoroughly using 0.5M NaOH and 400 ppm NaOCl at 50° C. The NWP was measured and shown to be recovered to >90% of the original NWP. Run numbers 081803A, 081803B, 081903A all show a process flux in the range of 110-130 LMH. These process fluxes correspond to the process flux of a new membrane as demonstrated by run 082003A.

The final fractionated product from each run was compared using SDS Page. Although the process flux was significantly reduced when using a fouled membrane, the fractionation does not appear to be greatly effected. This data supports the NWP recovery model previously described is valid and may be used for this fractionation process. v,18-20/2

According to the current invention the objective of separating the protein of interest from contaminating proteins using HPTFF was demonstrated. The goal of this fractionation was to retain the target protein (AFP) and pass the major contaminating milk proteins. The RPC, total protein, and SDS gel results conclusively showed the contaminating milk proteins could be effectively reduced in concentration. All but the recombinant human alphafetoprotein and CSA are effectively reduced in concentration using a 100 kD tangential flow membrane and methods of the invention.

Membrane Molecular Weight Cutoff (MWCO)

The membrane pore size and chemistry plays a considerable role in the effectiveness of the fractionation. Several MWCO membranes were evaluated including a 30 KD, 50 KD, 70 KD and 100 KD. If the MWCO is to low as in the case of the 30 KD, the contaminating proteins are retained no matter what operating conditions are used. A fractionation using this pore size would not effectively fractionate the contaminating proteins from the recombinant human alphafetoprotein protein. Each larger pore size was evaluated for its retention qualities and selectivity. The 30 kd, 50 kd, and 70 kd membranes all proved to retain many of the contaminating milk proteins and could not efficiently be used for this fractionation. The 100 kd membrane initially showed substantial product loss before being optimized. The pore size of this membrane proved to be the largest size able to be used, yet still be able to retain the recombinant human alphafetoprotein protein.

Various membrane types including regenerated cellulose, polyacrylonitrile (PAN), and modified polyethersulfone (PES). Each membrane has its own unique set of properties and can influence the fractionation. Once the proper membrane pore size was chosen, the membrane type or chemistry was be evaluated. The Pall Corp. Omega modified polyethersulfone (PES) membrane was chosen for its uniform pore size and neutral membrane charge.

Transmembrane Pressure (TMP)

The optimal Transmembrane pressure determined using the 100 KD HPTFF membrane in a re-circulation mode. The starting material was clarified milk diafiltered with 20 mM Phosphate buffer at pH 6.5. The buffer conditions suited this fractionation as low salt concentration increases the retention of the rhAFP molecule. The optimization curve showed the process TMP should be approximately 12-20 psi, just into the transition zone. A lower TMP, in the membrane controlled region, amplifies charge interaction between the contaminating milk proteins and the membrane reducing their transmission. A higher TMP, in the gel layer controlled region, drives all of the proteins to the surface and increases the mass transfer coefficient of the bulk solution. This leads to low recovery of the recombinant human alphafetoprotein protein and a less efficient fractionation. The cross flow velocity was not further optimized, but rather held at 0.6 L/min/ft2, recommended by the manufacturer.

Clarified Milk pH and Ionic Strength

The isoelectric point of the recombinant human alphafetoprotein is approximately 5.0 and the isoelectric point of the membrane is 7.0. In order to maximize the retention of the recombinant human alphafetoprotein, the ph of the buffer solution was chosen to be between 6.0-6.5. Under these conditions, both the membrane and recombinant human alphafetoprotein molecule will have a negative charge and repel each other. The ionic strength played a significant role in the retention properties of the recombinant human alphafetoprotein protein molecule. Under conditions where the salt conditions were elevated (>1.0 ml NaCl) the molecule passed more freely through the pores of the 100 kd membrane under most all operating conditions. The opposite is true under reduced salt conditions; the majority of the recombinant human alphafetoprotein protein molecule is retained. Two factors are known to be contributing to this phenomenon. The reduction of the salt concentration increases charge affects between the protein and the membrane. Additionally the reduction in salt concentration causes a “swelling” effect of the water layer surrounding the recombinant human alphafetoprotein protein. These combined effects cause the molecule to be retained by the 100 KD membrane where it would normally not be.

Clarified Milk Concentration Factor (CFac)

The concentration factor of the clarified milk is initially 2 times the concentration of the whole milk. The clarified milk is then fractionated using the 100 KD HPTFF membrane system of the invention. Typically the optimal point of diafiltration (CD) is determined by multiplying the value of the maximum gel layer concentration (CG) times CG/e.
C_{D}=C_{G}/e=0.37 C_{G}

The optimal concentration for the diafiltration portion of the fractionation is not easily calculated as an appreciable amount of the protein is removed as the clarified milk is concentrated. The CG is therefore always changing as the bulk protein concentration does not increase as predicted on a semi-log plot.

The optimal point of diafiltration was therefore arrived at experimentally. The clarified milk was diafiltered at 1×, 2×, and 4×. The effect of increasing the concentration factor before fractionating increased the number of diafiltration volumes required to reach the same level of purity. The benefit to concentrating 4× however was that a 50% reduction in buffer requirements could be achieved even though the number of diafiltration volumes doubled.

Number of Diafiltration Volumes

The number of diafiltration volumes used to fractionate the recombinant human alphafetoprotein was determined by the point at which the amount of contaminating proteins being removed was less than the amount of recombinant human alphafetoprotein being lost in the permeate. That point can be estimated by observing the SDS PAGE information. The initial number of diafiltration volumes needed at 1× concentration was approximately 10DV's and at 4× was 20DV's. The most efficient scheme proved to be the later at 4× concentration with 20DV's.

Changes to the diafiltration endpoint were made following the initial Q-Column development. The amount of total protein was monitored from run to run and proved to be a more effective way to predict the correct number of diafiltration volumes to use.

The final method chosen for the fractionation was to diafilter until the retentate falls below an O.D. measured at 280 nm. The exact point at which to stop the diafiltration was determined by the absorbance of the retentate at 280 nm. The target absorbance was found to be 4.0 AU at 4× concentration. This absorbance of 4.0 at 280 nm roughly corresponds to 6 mg/ml of total protein by RPC. This allows the process to consistently produce a fractionated product at the same total protein concentration regardless of the starting concentration. This concentration of total protein was chosen due to restraints put on the process by the subsequent anion exchange chromatographic step. The ratio of contaminating protein to AFP affects the loading of the Q-column and must therefore be consistent from run to run. The target set for the final total protein concentration of the Q-Load is 2.8-3.2 mg/ml once diluted 1:1 with 20 mM Phosphate buffer.

Clarified Milk Lot

The lot of clarified lot used for the fractionation has an impact on the amount of diafiltration required to reach the same endpoint (O.D.@280 nm). The clarified milk collected earlier in the lactation tended to have 50% more total protein than lots collected at the end of the lactation. The amount of product in the clarified lots tended to remain constant however. The difference in protein concentration was likely due to higher amounts of contaminating milk proteins early in the lactation. These early lots of clarified milk in turn required approximately 50% more diafiltration than later ones. Analysis which included RPC, SDS PAGE, and Bradford total protein test all shows similar results between all of the fractionated lots regardless of the clarified material used.

Membrane Recovery—Normalized Water Permeability (NWP)

HPTFF cassette systems are designed to be re-used for periods of time up a year. It is therefore important that the membrane be effectively cleaned following each run. The cleaning solution used for the 100 KD membrane was 0.5M NaOH and 400 ppm bleach at 40-50 C. for 1 hour. This cleaning regime proved to be effective in recovering the membrane's normalized water permeability (NWP). The membrane's NWP is considered to be “recovered” if it is within 90% of the original NWP taken when the cassette was new. The graph of cycle# vs. NWP shows the membrane should have a projected life of at least 60 runs.

To further prove the protein fractionation is robust and membrane recovery can predict membrane performance, a membrane was purposely fouled and then used three times for the protein fractionation. The process flux was approximately half of its clean counterpart, but the fractionation efficiency was surprisingly unchanged. The fractionated material was analyzed using RPC and SDS PAGE to show uniformity. This data shows the water flux can be well below the specification of 90% recovery that has been put on it.

The data generated from this experiment showed that protein fractionation using a 100 kD MWCO, HPTFF system could of the invention effectively reduce the concentration of contaminating milk proteins. The process operates efficiently with a liquid flux of 80 to 100 LMH and requires a minimal amount of capital equipment when compared to other upstream purification methods such as chromatography. The relative recombinant human alphafetoprotein protein purity initially begins between 6-10% in the clarified milk and is increased to 30% purity following the fractionation. The process yield is consistently in the 80% range and is comparable to subsequent purification steps.

Pursuant to the current invention the experimental strategy was to determine the relationships between the filtration process variables that can be controlled on a large scale, (CM, V, TMP, T), where V is Flow Velocity, as can product passage, retention and quality. The relationships were established through a matrix of individual bench scale experiments, and optimal windows of operation were identified. These optimal parameters are combined into a experimental series where overall yield and mass balance are investigated. Performance was determined by product yield, clarity, and flux efficiency. The following process variables are investigated in the individual bench scale experimental matrix.

Concentration (Cm) Optimal milk concentration factors were be determined with empirical product passage data. The rate of product passage per meter squared in a fixed time is referred to as the product flux (Jp). Product flux will be measured in relationship to concentration factor during the Clarification step (Unit Operation # 1).

Again referring to FIG. 1, below is provided an explanation of the elements of the invention.

FIG. 1 Elements

Process Stream Description

| Stream | |
|---|---|
| Number | Description |
| 1a | Raw tg Milk |
| 1b | Microfiltration CIP Solutions |
| 2a | Microfiltration Retentate to drain after Diafiltration |
| 2b | Used CIP Solutions to drain |
| 3 | In process MF Retentate (loop) |
| 4 | MF CIP Recirculation (loop) |
| 5 | Microfiltration Filtrate |
| 6 | Ultrafiltration CIP Solutions |
| 7 | Used CIP Solutions to drain |
| 8 | Ultrafiltration Feed (Microfiltration Filtrate) |
| 9 | In process UF Retentate (loop) |
| 10 | Ultrafiltration Permeate (To Diafilter MF Retentate) |
| 11 | Concentrated Clarified Bulk |
| 12 | UF CIP Recirculation (loop) |
| 13 | AF CIP Solutions |
| 14 | Aseptic Filter Feed |
| 15 | Bioburden Reduced Concentrated Clarified Bulk |
| 16 | Used CIP Solutions to drain |

In its broadest aspect, the high-performance tangential-flow filtration process contemplated by the invention provided herein involves passing the mixture of the species to be separated through one or more filtration membranes in an apparatus or module designed for a HPTFF type of system under certain conditions of TMP and flux. The TMP is held at a range in the pressure-dependent region of the flux v. TMP curve, namely, at a range that is no greater than the TMP value at the transition point. Thus, the filtration is operated at a flux ranging from about 5% up to 100% of transition point flux. See Graphs. A and B below, wherein the flux v. TMP curve is depicted along with the transition point. As a result, the species of interest are selectively retained by the membrane as the retentate while the smaller species pass through the membrane as the filtrate, or the species of interest pass through the membrane as the filtrate and the contaminants in the mixture are retained by the membrane. It should be noted that the species of interest for ultrafiltration preferably are biological macromolecules having a molecular weight of at least about 1000 daltons, and most preferably polypeptides and proteins. Also preferred is that the species of interest be less than ten-fold larger than the species from which it is to be separated, i.e., contaminant, or be less than ten-fold smaller than the species from which it is to be separated.

As used herein, the expression “means for re-circulating filtrate through the filtrate chamber parallel to the direction of the fluid in the filtering chamber” refers to a mechanism or apparatus that directs a portion of the fluid from the filtrate chambers to flow parallel to and in substantially the same direction (allowing for some eddies in flow to occur) as the flow of fluid passing through the adjacent filtering chamber from the inlet to the outlet of the filtering chamber. Preferably, this means is a pumping means.

It is noted that the TMP does not increase with filtration time and is not necessarily held constant throughout the filtration. The TMP may be held approximately constant with time or may decrease as the filtration progresses. If the retained species are being concentrated, then it is preferred to decrease the TMP over the course of the concentration step.

Each membrane preferably has a pore size that retains species with a size of up to about 10 microns, more preferably 1 kDa to 10 microns. Examples of species that can be separated by ultrafiltration include proteins, polypeptides, colloids, immunoglobulins, fusion proteins, immunoglobulin fragments, mycoplasm, endotoxins, viruses, amino acids, DNA, RNA, and carbohydrates. Examples of species that can be separated by microfiltration include mammalian cells and microorganisms such as bacteria.

Because membrane filters are not perfect and may have holes or irregularities that may be large enough to allow some intended retentate molecules to slip through, a preferred aspect herein is to utilize more than one membrane having the same pore size, where the membranes are placed so as to be layered parallel to each other, preferably one on top of the other. Preferably the number of membranes for this purpose is two.

While the flux at which the pressure is maintained in the above process suitably ranges from about 5 to 100%, the lower the flux, the larger the surface area of the membrane required. Thus, to minimize membrane cost, it is preferred to operate at a pressure so that the flux is at the higher end of the spectrum. The preferred range is from about 50 to 100%, and the more preferred range is about 75 to 100%, of the transition point flux.

While the TMP need not be maintained substantially constant along the membrane surface, it is preferred to maintain the TMP substantially constant. Such a condition is generally achieved by creating a pressure gradient on the filtrate side of the membrane. Thus, the filtrate is recycled through the filtrate compartment of the filtration device in the same direction and parallel to the flow of the mixture in the retentate compartment of the device. The inlet and outlet pressures of the recycled material are regulated such that the pressure drop across the filtrate compartment equals the pressure drop across the retentate compartment.

Several practical means can be used to achieve this filtrate pressure gradient. Some examples of preferred embodiments are the configurations shown in FIGS. 2A and 2B. According to these configurations the solutes to be separated enter the device through an inlet conduit 36, which communicates with a fermenter tank (not shown) if the products to be separated are in a fermentation broth. It may also communicate with a vessel (not shown) that holds a source of transgenic (Tg) milk or cell lysate or a supernatant after cell harvest in cell culture systems. The flow rate in conduit 36 is regulated via a pumping means 40. The pump is any suitable pump known to those skilled in the art, and the flow rate can be adjusted in accordance with the nature of the filtration as is known to those skilled in the art.

In a Microfiltration Unit 30 of the current invention, a pressure gauge 45 is optionally employed to measure the inlet pressure of the flow from the pumping means 40. The fluid in inlet conduit 36 enters filtration unit 50. This filtration unit 50 contains a filtering chamber 51 in an entrance top portion thereof and a filtrate chamber 52 in the exit portion. These two compartments are divided by a filtration membrane 55. The inlet fluid flows in a direction parallel to filtration membrane 55 within filtering chamber 51. The upper, filtering chamber 51 receives the mixture containing the solute containing a molecule of interest of interest. Molecules small that the target molecule are able to pass through the membrane 55 into the filtrate or exit chamber 52. The concentrated retentate passes from the filtration unit 50 via outlet conduit 60, where it may be collected and processed further by a microfiltration (MF) membrane 65, if necessary, to obtain the desired species of interest including moving through an additional membrane. During this entire process, and for quality control purposes, a series of sample points 99 are contemplated by the current invention to allow monitoring of molecule concentration, pH and contamination—“path B”. Alternatively, a retentate stream is circulated back to a tank or fermenter 35 “path A” from whence the mixture originated, to be recycled through inlet conduit 36 for further purification.

A solution containing molecules of interest that pass through the membrane 55 into the filtrate chamber 52 can also leave filtration unit 50 via outlet conduit 70 at the same end of the filtration unit 50 as the retentate fluid exits via outlet conduit 60. However, the solution and molecules of interest flowing through outlet conduit 70 are sent back to tank 35, and are measured by pressure gage 72 for further processing.

Similarly, and as depicted in FIG. 2B a Dual TFF system 80 according to the current invention is contemplated.

In the configuration shown in FIG. 2A, the membranes will need to be placed with respect to chambers 51 and 52 to provide the indicated flow rates and pressure differences across the membrane. The membranes useful in the process of this invention are generally in the form of flat sheets, rolled-up sheets, cylinders, concentric cylinders, ducts of various cross-section and other configurations, assembled singly or in groups, and connected in series or in parallel within the filtration unit. The apparatus generally is constructed so that the filtering and filtrate chambers run the length of the membrane.

Suitable membranes are those that separate the desired species from undesirable species in the mixture without substantial clogging problems and at a rate sufficient for continuous operation of the system. Examples include microporous membranes with pore sizes typically from 0.1 to 10 micrometers, and can be made so that it retains all particles larger than the rated size. Preferably they are ceramic for both microfiltration uses and TFF uses according to the current invention. Ultrafiltration membranes have smaller pores and are characterized by the size of the protein that will be retained. They are available in increments from 1000 to 1,000,000 Dalton nominal molecular weight limits.

Ultrafiltration membranes are most commonly suitable for use in the process of this invention. Ultrafiltration membranes are normally asymmetrical with a thin film or skin on the upstream surface that is responsible for their separating power. They are commonly made of regenerated cellulose or polysulfone.

Membrane filters for tangential-flow filtration system 80 are available as units of different configurations depending on the volumes of liquid to be handled, and in a variety of pore sizes. Particularly suitable for use in the present invention, on a relatively large scale, are those known, commercially available tangential-flow filtration units.

In an alternative and preferred apparatus, and for the reasons presented above, the microfiltration unit 30 of FIG. 2A comprises multiple, preferably two, filtration membranes, as membranes 56 and 57, respectively. These membranes are layered in a parallel configuration.

The invention also contemplates a multi-stage cascade process wherein the filtrate from the above process is passed through a filtration membrane having a smaller pore size than the membrane of the first apparatus in a second tangential-flow filtration apparatus, the filtrate from this second filtration is recycled back to the first apparatus, and the process is repeated.

One tangential-flow system 80 suitable for process according to the invention or use in conjunction with a microfiltration unit 30 is shown in FIG. 2B. Here, a first vessel 85 is connected via inlet conduit 90 to a filtering chamber 96 disposed within a filtration unit 95. A first input pumping means 100 is disposed between the first vessel 85 and filtering chamber 96. The filtering chamber 96 is connected via an outlet conduit 110 to the first vessel 85. The filtering chamber 96 is separated from a first filtrate chamber 97 situated directly below it within filtration unit 95 by a first filtration membrane 115. The first filtrate chamber 97 has an outlet conduit 98 connected to the inlet of chamber 97 with a filtrate pumping means 120 disposed in the conduit 98. Conduit 45, which is connected to outlet conduit 98, is connected also to a second vessel 120.

This vessel 120 is connected via inlet conduit 125 to a second filtering chamber 127 disposed within a second filtration unit 130. A second input pumping means 133 is disposed between the second vessel 120 and filtering chamber 127. The filtering chamber 127 is separated from the second filtrate chamber 129 situated directly below it within filtration unit 130 by a second filtration membrane 128. The second filtrate chamber 129 has an outlet conduit 135 connected to the inlet of chamber 129 with a filtrate pumping means 140 disposed in the conduit 135. Conduit 125, which is connected to outlet conduit 135, is connected also to a third vessel 150.

This vessel 150 is connected via inlet conduit 155 to a third filtering chamber 157 disposed within a third filtration unit 160. A third input pumping means 165 is disposed between the third vessel 150 and filtering chamber 157. The filtering chamber 157 is separated from the third filtrate chamber 159 situated directly below it within filtration unit 160 by a third filtration membrane 165. The third filtrate chamber 159 has an outlet conduit 170 connected to conduit 155, which is connected to first vessel 150, to allow the filtrate to re-circulate to the original tank. Sample points 99 were also provided for monitoring the process, as well as pressure gages 175.

The process of the present invention is well adapted for use on a commercial scale. It can be run in batch or continuous operations, or in a semi-continuous manner, e.g., on a continuous-flow basis of solution containing the desired species, past a tangential-flow filter, until an entire large batch has thus been filtered, with washing steps interposed between the filtration stages. Then fresh batches of solution can be treated. In this way, a continuous cycle process can be conducted to give large yields of desired product, in acceptably pure form, over relatively short periods of time.

The unique feature of tangential-flow filtration as described herein with its ability to provide continuous filtration of solids-containing solutions without filter clogging results in a highly advantageous process for separating and purifying biological reaction products for use on a continuous basis and a commercial scale. Moreover, the process is applicable to a wide range of biological molecules, e.g., protein products of transgenic origin, antibodies, cell fragments and cell culture lysates.

The following examples illustrate the invention in further detail, but are not intended to be limiting. In these examples, the disclosures of all references cited are expressly incorporated by reference.

Clarification Modules

Membranes useful in the current invention can be fabricated into production modules in several formats. The most common formats used for tangential flow filtration are:
Flat plate
Spiral wound
Hollow fiber

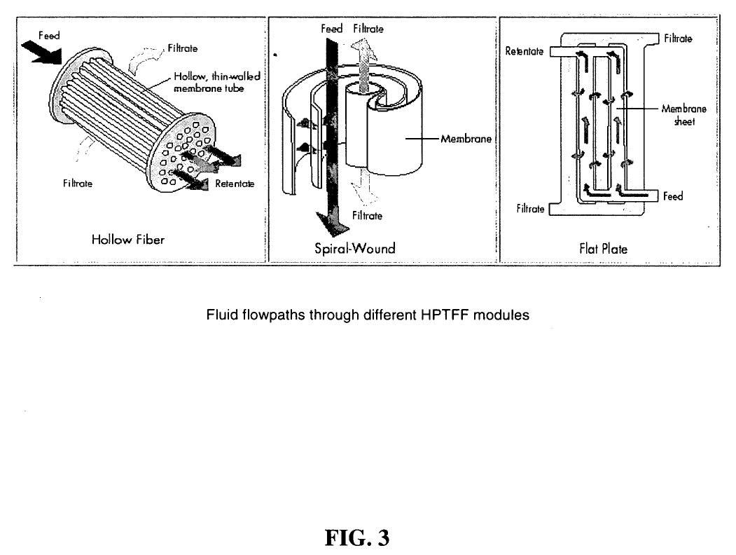

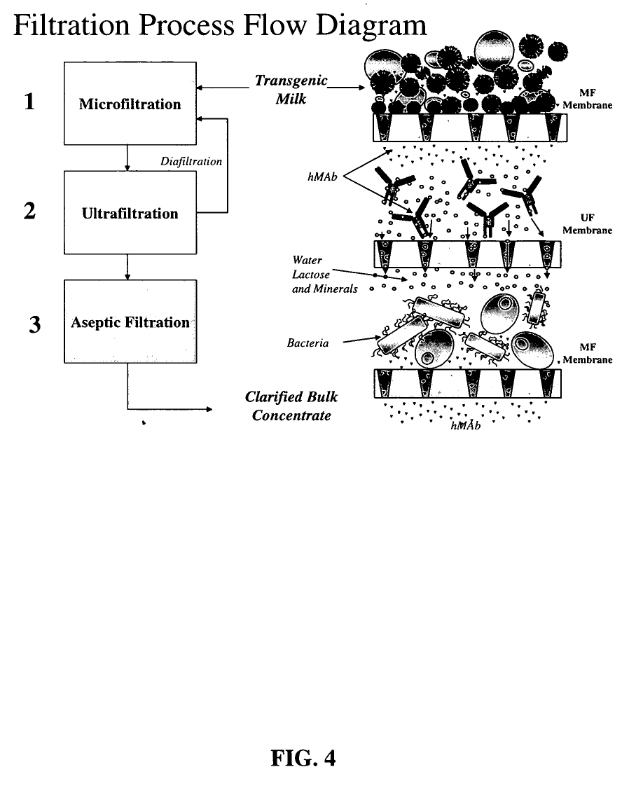

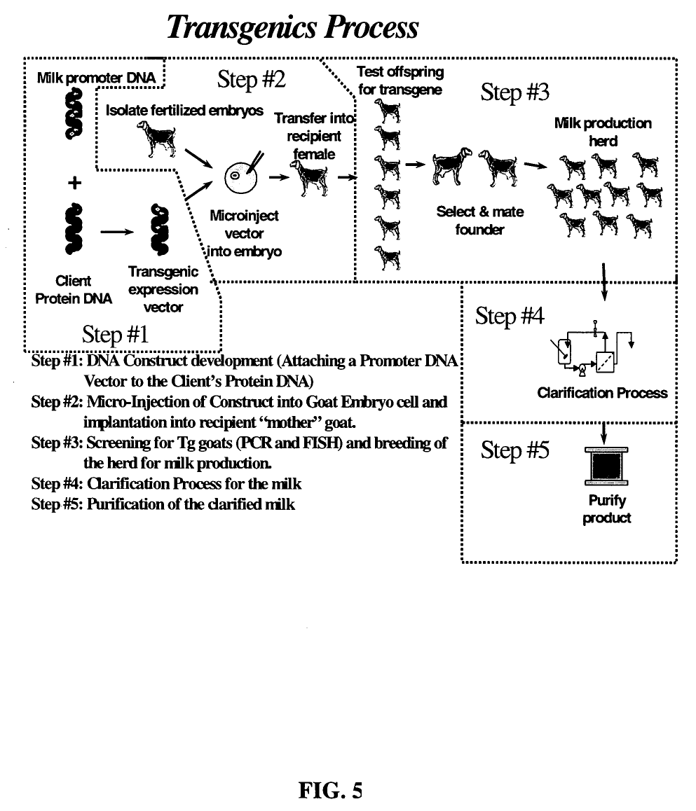

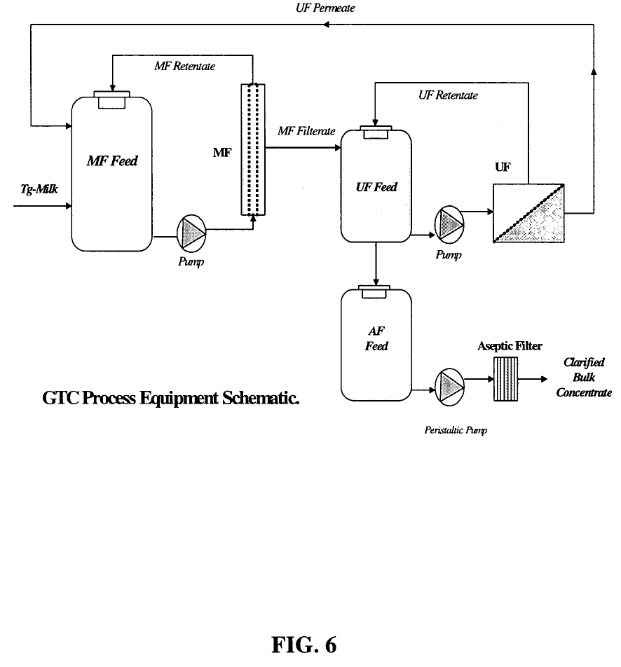

The basic flowpaths for each of these modules is shown in FIG. 3, which demonstrates the fluid flowpaths for a feedstream through different HPTFF and TFF modules.

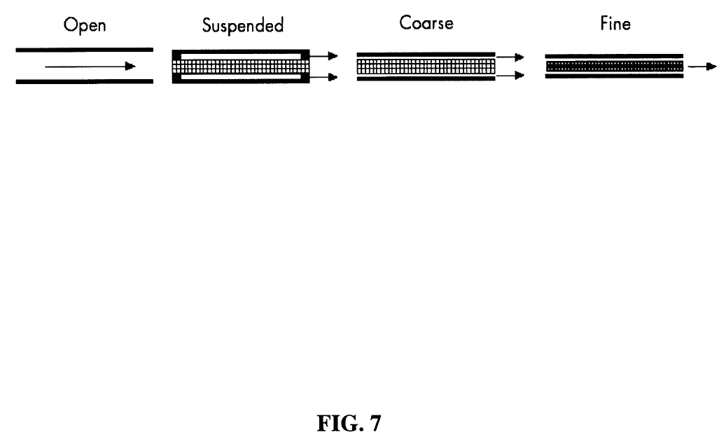

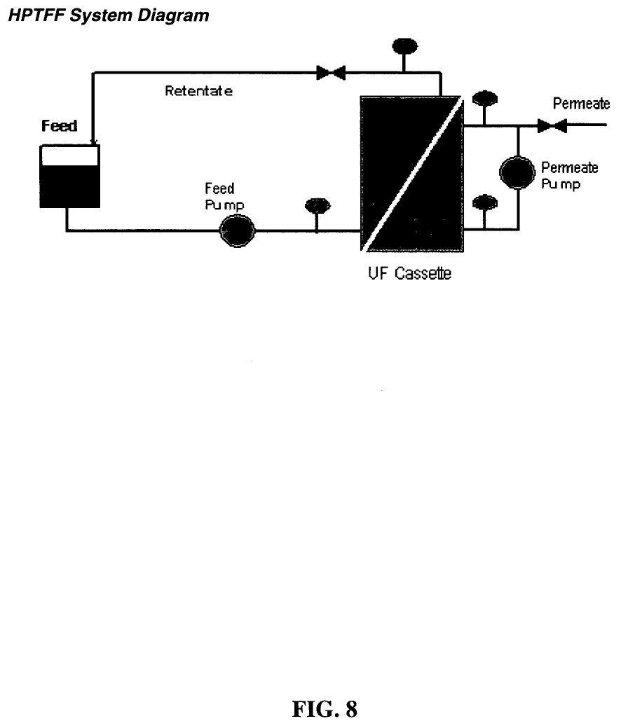

Screens are often inserted into the feed and/or filtrate channels in spiral wound and flat plate modules to increase turbulence in the channels and reduce concentration polarization. This is not an option with hollow fiber modules. The turbulence-promoted channels have higher mass transfer coefficients at lower crossflow rates, meaning that higher fluxes are achieved with lower pumping requirements. Turbulence-promoted feed channels are, therefore, more efficient than open channels. Using a suspended screen in a flat plate module gives some of the benefits of both open and turbulence-promoted channels. FIG. 7 illustrates the different types of channel configurations.

Flat Plate

(Often referred to as Cassettes) In a flat plate membrane module, layers of membrane either with or without alternating layers of separator screen are stacked together and then sealed into a package. Feed fluid is pumped into alternating channels at one end of the stack and the filtrate passes through the membrane into the filtrate channels. Flat plate modules generally have high packing densities (area of membrane per area of floor space), allow linear scaling, and some offer the choice of open or turbulence promoted channels.

Spiral Wound

In a spiral wound module, alternating layers of membrane and separator screen are wound around a hollow central core. The feed stream is pumped into one end and flows down the axis of the cartridge. Filtrate passes through the membrane and spirals to the core, where it is removed. The separator screens increase turbulence in the flowpath, leading to a higher efficiency module than hollow fibers. One drawback to spiral wound modules is that they are not linearly scaleable because either the feed flowpath length (cartridge length) or the filtrate flowpath length (cartridge width) must be changed within scales.

Hollow Fiber

Hollow fiber modules are comprised of a bundle of membrane tubes with narrow diameters, typically in the range of 0.1 to 2.0 mm. In a hollow fiber module, the feed stream is pumped into the lumen (inside) of the tube and filtrate passes through the membrane to the shell side, where it is removed. Because of the very open feed flowpath, low shear is generated even with moderate crossflow rates. While this may be useful for highly shear-sensitive products, in general it reduces the efficiency of the module by requiring very high pumping capacity to achieve competitive fluxes.

For all experiments conducted with the microfiltration system except a feed-and-bleed experiment, the equipment used was the following:
60 lpm pump calibrated to correlate pump (Pump Curve)
   1″ OD stainless steel sanitary piping
   0.2 um pore size ceramic membrane of either 0.2 sqft or 1.5 sqft
   Stainless steel sanitary membrane holder with one ½″ outlet port
   ¼″ ID flexible permeate tubing
   Diaphragm valve on the retentate line
   2 pressure gauges
   Steel 1.2 L feed reservoir
   ¾″ ID flexible retentate tubing.

For all HPTFF experiments, the preceding equipment was coupled with the following equipment:
Diaphragm pump with maximum output of 800 mLPM
¼″ ID flexible pressure resistant tubing on all lines
1 pressure gauge for feed pressure measurements
2 diaphragm valves on the retentate and permeate lines
30 kDa NMWCO PES Pall Filtron Centramate membrane of either 0.2 sqft or 1 sqft
Stainless steel Pall Filtron Centramate membrane holder
1 stainless steel u-bend pipe to connect permeate ports.
   Membrane Selection

The membranes selected for the HPTFF system of the invention were selected from a group of membranes of varying geometries and nominal molecular weight cut-offs. Previous studies explored the use of polymeric based high MWCO UF membranes, as well as ceramics, for the clarification step. Concentrating the milk down 2× and then doing HPTFF challenged all membranes. The membranes were then analyzed for reusability by challenging them with multiple runs and cleanings. A membrane was considered recovered for the next process when the normalized water flux was maintained above 80% of the virgin membrane. None of the flat sheet polymeric membrane cassettes maintained the target water flux recovery after 3 uses, while the ceramic membrane was recovered more than 60 times. This was due to the ability to clean the ceramic using harsher conditions of higher chemical concentration and higher temperatures. The 30 kDa ultrafiltration membrane maintained high water flux recoveries beyond 20 cycles.

The first unit, used to clarify the milk and pass a protein of interest, was tested using 0.2 um nominal ceramic tubular membranes. The second system used to capture the protein of interest was tested with flat sheet ultrafiltration membranes of 30 kDa molecular weight cut-offs.

Analytical Methods

Samples from each experiment samples were analyzed for recombinant human alphafetoprotein (rhAFP) content by protein A HPLC, for degradation by SDS-PAGE, for modification by isoelectric focusing (IEF), and for aggregation by size exclusion chromatography (SEC).

Procedure

A series of controlled experiments were conducted employing 0.2 μm molecular weight cut-off ceramic microfiltration membranes in the hopes of understanding process operational relationships. Product Flux (Jp) was measured as it related to flow velocity (u), trans-membrane pressure (TMP), temperature (t), and milk concentration (c). Once relationships were established, optimal windows of operation were determined and a compiled process was tested. Samples were taken and mass balance data was gathered and analyzed for initial product yield and throughput. (Please see, FIGS. 2A and 2B).

Temperature Experiment

The objective was to determine the range of operating temperatures which give optimum rhAFP flux at lowest volume through a 0.2 um, 3 mm channel ceramic MF membrane. To analyze rhAFP degradation by SDS-PAGE and Western blot during processing the pH of each milk segment was taken prior to milk pooling. The milk is pooled into the MF feed tank and total volume is recorded. The MF pump controller is ramped up from 20 Hz to 45 Hz (approximately 5 L/min to approximately 20 L) at this time. All parameters at every successive time point are recorded such as temperature, pressures, cross-flow rate, permeate flow rate, and volume. This MF loop is run in recirculation (path A) for 5 minutes. The transmembrane pressure is adjusted to 12 psig and re-circulated (path A) for 5 minutes (Maintained a temperature of 20° C). The permeate line is directed to drain until milk was concentrated 2× the original milk volume (permeate was collected). Temperature was maintained at 20° C. Samples 2 and 3 were taken from the feed reservoir and from the permeate line. The permeate line was then returned to path A and re-circulated for 10 minutes. Samples 4 and 5 were taken. Temperature was allowed to increased to 25° C. The system then re-circulated for 10 minutes and samples 6 and 7 were taken. Temperature was allowed to increased to 30° C. The system then re-circulated for 10 minutes and samples 8 and 9 were taken. Temperature was allowed to increased to 35° C. The system then re-circulated for 10 minutes and samples 10 and 11 were taken. Temperature was allowed to increased to 40° C. The system then re-circulated for 10 minutes and samples 12 and 13 were taken. The pump was then turned off and samples were stored at 2-8° C. and sent for quantitation. Samples were analyzed by IEF.

MF Milk Concentration Experiment

The objective of this experiment according to a preferred embodiment of the invention was to determine the range of initial milk concentration which gives optimum protein of interest flux at lowest volume through a 0.2 um, 3 mm channel ceramic MF membrane.

In terms of procedure the pH of each milk segment was taken prior to milk pooling. The milk is pooled into the MF feed tank and total volume is recorded. The MF pump controller is ramped up from 20 Hz to 45 Hz (approximately 5 L/min to approximately 20 L) at this time. All parameters at every successive time point are recorded such as temperature, pressures, cross-flow rate, permeate flow rate, and volume. This MF loop is run in recirculation (path A) for 5 minutes. The transmembrane pressure is adjusted to 12 psig and re-circulated (path A) for 5 minutes (Maintained a temperature of 20° C). Adjusted transmembrane pressure to 15 psig and re-circulated (path A) for 5 minutes. The permeate line was directed to drain until milk was concentrated, and 550 ml of permeate was collected, then returned the permeate line to path A.(Re-circulated for 10 minutes) Samples 2 and 3 were taken from the feed reservoir and the permeate line respectively.

The permeate line was directed to path B and 600 ml of milk was added to the feed reservoir. The permeate line was directed to drain until milk was concentrated, and 500 ml of permeate was collected, then returned the permeate line to path A. (Re-circulated for 10 minutes) Samples 4 and 5 were taken from the feed reservoir and the permeate line respectively. The permeate line was then directed to path B and 500 ml of milk was added to the feed reservoir. The permeate line was directed to drain until milk was concentrated, and 500 ml of permeate was collected, then returned the permeate line to path A.(Re-circulated for 10 minutes) Samples 6 and 7 were taken from the feed reservoir and the permeate line respectively. The permeate line was then directed to path B and 380 ml of milk was added to the feed reservoir. The permeate line was directed to drain until milk was concentrated, and 400 ml of permeate was collected, then returned the permeate line to path A.(Re-circulated for 10 minutes) Samples 8 and 9 were taken from the feed reservoir and the permeate line respectively. The pump was then turned off. Samples were stored at 2-8° C. and sent for protein of interest quantitation by protein A analysis, SDS-PAGE and Western for degradation and aggregation, SEC for aggregation, and IEF for isoelectric point shifts.

HPTFF was implemented as a process to clarify and stabilize rhAFP in a milk matrix by removing particulate matter such as fat, casein micelles, and bacteria from raw milk. HPTFF is used in a limited fashion in both the biotechnology and dairy industries to remove impurities and concentrate product. According to the current invention, in order to use HPTFF effectively it is important that the proper membranes are chosen, the process parameters (temperature, trans-membrane pressure, cross-flow velocity, and milk concentration) are optimized for high product flux, and the cleaning and storage procedures were developed to ensure long membrane life. Experimental matrix parameters are described herein, according to the current invention and applied to transgenic goat milk to confirm previous operational parameters. Membrane cleaning and storage conditions were also investigated. An aseptic filtration step was developed to remove any bacteria remaining from the clarified milk product containing a protein of interest after the HPTFF process is complete. Process information was then transferred to pilot scale equipment were initial engineering runs were conducted. Some process design criteria included, using no additives to prevent the need for water for injection, long membrane life, high yield, and short processing time. The process of the current invention was preferably designed to be scalable for pilot and manufacturing operations.

Non-Transgenic Feed-And-Bleed Experiment

Non-transgenic milk was used to analyze liquid flux decay during concentration using the 0.2 um ceramic microfiltration membrane since an abundant supply of non-transgenic milk is available. The equipment used for this experiment included the same equipment described for microfiltration experiments, but it was supplemented by a second feed reservoir and a feed pump to flow milk into the feed reservoir of the microfiltration system at the same rate that permeate was flowing out of the membrane.

The equipment schematic is: v,21/2

As seen in Graph A, the feed reservoir was filled with 1500 ml of milk and the pump was started at 45 Hz. The system was run in re-circulation for 10 minutes with no retentate pressure. All parameters were recorded. The retentate pressure was then increased to 10 psig for a transmembrane pressure of 11 psig. This transmembrane pressure was held constant throughout the experiment by adjusting the retentate valve. The permeate was sent to drain, and a second pump was started up to pump fresh milk into the feed reservoir at the same rate as permeate was removed, keeping the volume in the feed reservoir constant. All parameters were recorded at 5-10 minute intervals, and the second pump speed was adjusted to keep the level of milk in the feed reservoir constant. The experiment was run until the milk was concentrated 5.37× or 82%.

Membrane Cleaning

A stringent cleaning regime was employed in order to assure high cycle to cycle membrane water flux recovery. Cleaning steps were designed to mimic standard membrane cleaning in the dairy industry taking into consideration aspects of biopharmaceutical practices. The water flush steps were optimized to minimize water use while flushing out residual chemical for proper pH and conductivity values. The following cleaning cycles were carried out after every processing step provided in Tables 1 and 2 below:

TABLE 5
Ceramic membrane cleaning steps:

| | | | | | |
|---|---|---|---|---|---|
| Step | Concentration | Volume | Time | Temp | pH |

| | | | | | | | |
|---|---|---|---|---|---|---|---|
| 1) | Water Flush | — | 16-20 | L | 5 min. | 60° C. | 7.0 |
| 2) | NaOH Wash | 0.5 M | 1 | | 10 min. | 60 | >11.5 |
| | Sodium Hypochlorite | 400 ppm | | | | | |
| 4) | NaOH Wash | 0.5 M | 1 | | 30 min. | 60 | >11.5 |
| | Sodium Hypochlorite | 400 ppm | | | | | |
| 5) | Water Flush | — | 20-25 | | 5 min. | 60 | 7.0 |
| 6) | Citric Acid Wash | 0.4 M | 1 | | 30 min. | 60 | <2.75 |
| 7) | Water Flush | — | 16 | | 10 min. | 60 | 7.0 |
| 8) | Sodium Hypochlorite | 300 ppm | 1 | | 15 min. | 60 | >9.5 |
| | NaOH | 0.05 M | | | | | |
| 9) | Water Flush | — | 12 | | 10 min. | 60 | 7.0 |
| 10) | NaOH Storage | 0.1 M | 1 | | | 20 | 10-12 |

After a number of engineering runs on the equipment used in the pilot plant to clarify milk, it was determined the equipment and procedures used required modification in order to produce clear clarified milk consistently. The equipment was removed from the GMP environment of the pilot plant to the development laboratory for extensive testing. The modifications made to the system included reducing the permeate piping and changing the location of the valves in the system to facilitate easier rinsing during the cleaning and sanitization steps. The cleaning protocols were slightly modified to improve the cleaning efficiency and reduce water usage. Process temperature ranges were determined. Finally, the process parameters were better defined in the GMP documentation.

The original design for the pilot equipment was constructed entirely of stainless steel. This design was cumbersome to clean since many long lengths of pipe needed to be disassembled from the process mode into the cleaning mode. Because of the length and inner diameter of the UF permeate piping, it was not effectively cleaned or rinsed during the cleaning protocol. A number of pieces were added to the MF system to facilitate cleaning, however their construction caused dead spaces for debris to accumulate. These problems were remedied by replacing the long UF permeate piping with ¼″ inner diameter tubing. The cleaning set-up was altered such that the top port of the MF membrane would be used for cleaning the permeate side of the membrane eliminating the need for the other pieces. The UF permeate tubing then remains on the UF during cleaning. Also, a large heat exchanger had been installed on the MF portion of the system, which allowed fine temperature control on the MF, but prevented controlling the UF temperature within the proper range for processing. The heat exchanger was removed from the system, and the chiller setting was adjusted to properly cool both systems within the proper temperature range. The final design is below. Equipment assembled for storage, sanitizing and processing. Configuration of equipment in an a preferred embodiment of the invention is provided in Graphs O and P below.

Cleaning and Sanitization Changes

The equipment changes performed necessitated altering the cleaning and sanitization protocols. The cleaning protocol was run after every run in the table above. The retentate valve on the MF needed to be left half-open to facilitate proper rinsing during each rinse step since there is a long dead leg between the valve and the reservoir. After run 4, the cleaning protocol was run and the water consumption was tracked (Notebook 10586). The water used in this experiment was verified after runs 5, 6, and 7, and was recommended for use in GMP processing. As was stated before, the equipment alterations also allow the system to be sanitized in process mode. This was tested. The USP water required to rinse the sanitant from the system was also determined.

Operation

The actual steps taken to perform milk processing using HPTFF are described in the following sections. These include the entire process from sanitizing the systems, to processing, to cleaning, and to storing. The procedures were used on the equipment in the development lab during runs 5-7 and produced clear clarified milk.

Sanitization

To perform HPTFF using a ceramic 0.2 um microfiltration membrane and a 30 kda ultrafiltration membrane to clarify and concentrate transgenic goat milk, the system must be sanitized with 0.1M sodium hydroxide. The equipment is assembled for sanitization and processing as above. 2L of 0.1M sodium hydroxide made with USP water is pumped through each system, with 15LPM of cross flow on the MF and 1LPM of cross flow on the UF. No retentate pressure is added to the MF, while 5 psi of pressure is added to the retentate of the UF. The permeate valves are completely open allowing the sodium hydroxide to re-circulate around the entire system. The re-circulation is done for 15 minutes, and then the solution is drained from the system through the bleed valves between the tanks and the pumps. USP water is used to rinse out the system by filling the tanks up completely with USP water whenever necessary. 1L of water is drained from each bleed valve. The retentate valves on the MF are half closed, and the permeate valve is directed completely to waste. The retentate and permeate valves on the UF are directed completely to waste. 12L of USP water is flushed through the MF retentate with a cross flow rate of 20 LPM. 4L of USP water is flushed through the MF permeate with a cross flow rate of 15-20 LPM and 6-8 psi of TMP. 7L of USP water is flushed through the UF retentate and permeate lines with a cross flow rate of 1 LPM, then the permeate is flushed with an additional 3L.

Using USP water (adding more if necessary), pump the MF at 20 LPM, increase the retentate pressure until the TMP of 15 psi is reached with no permeate pressure, then adjust the cross flow rate with pump speed to 15 LPM. Record the temperature (must be between 25-28° C.), pressures, and cross flow rate. Measure the permeate flow rate through the permeate drain valve. Repeat on the UF using 1 LPM of cross flow, and 5 psig of retentate pressure, and no permeate pressure (TMP of approximately 10 psig). Compare the permeate flow rates to those of the membranes' virgin water permeability. If the permeation rate is less than 80% of the original value, either re-clean the membranes or replace them.

Milk Processing

The milk must be pooled and raised to 15-20° C. The milk is pooled in the MF reservoir, then the MF permeate valve is closed, the retentate valve is opened, and the pump is turned on for a cross flow of 20 LPM. After 5 minutes the initial milk sample(s) are taken. The pressure is then increased for a TMP of 15 psig and cross flow rate of 15 LPM. The re-circulation continues until the milk temperature reaches 20° C. Then the chiller is turned on at 10° C. and the MF permeate valve is opened to allow the milk to be concentrated to half of it's original volume on the microfiltration system by collecting the permeate of the ceramic membrane. The MF is run at 15 lpm cross flow rate with 15 psi of transmembrane pressure. The temperature of the MF should increase to and remain at 26° C.±2.0. The ultrafiltration system must then be started up at 0.8-1 LPM/sqft cross flow rate. The permeate flow rates of each membrane are measured through the permeate valves. The retentate and permeate pressures of the UF must be adjusted to cause the permeate flow rate to match the permeate flow rate of the MF. Once the UF permeate flow rate matches that of the MF. The systems should be run coupled for 5-6 diafiltration volumes.

Once diafiltration is complete, the systems are disconnected, the MF is shut of, drained and cleaned, and the UF permeate is directed to drain until the volume of bulk clarified concentrate in the feed reservoir of the UF is concentrated to half it's volume for a total concentration of 4×. The UF is then drained, the bulk clarified concentrate is aseptically filtered, and the UF is cleaned.

Cleaning and Storing Protocols

To appropriately clean and store the elements of the current apparatus that allow the fractionation of a protein of interest, first the systems are disconnected from feedstream inputs. The MF is rinsed with 20 L hot soft water (45-65° C.) with the retentate valves half open, and the permeate directed to drain. The valves are directed to re-circulate solution back to the feed reservoir, and 2 L of hot 0.5 M sodium hydroxide with 400 ppm sodium hypochlorite is re-circulated for 5 minutes. The solution is drained from the system and replaced with 2 L of the same chemicals. The fresh solution is re-circulated for 30 minutes, then drained through the bleed valve. The system is flushed with 20 L of hot soft water through the half opened retentate valves. 4 L is flushed through the permeate only by recirculating the water on the retentate side of the membrane at 20 lpm with 6-8 psi of TMP. Remaining water is drained through the bleed valve. 2 L of hot 0.5 M citric acid is re-circulated through the system for 30 min at 20 LPM with 6-8 psi of TMP. The citric acid is then drained out through the bleed valve. 15 L of soft water is used to rinse out the retentate side of the MF, and 4 L is used to rinse out the permeate side as was done after the caustic step. 2 L of hot 0.05 M sodium hydroxide with 400 pm bleach was then re-circulated through the MF for 15 minutes and drained and rinsed out with 10 L of water on the retentate side and 4 L through the permeate as was done after the caustic step.

The UF retentate and permeate lines are directed to drain for the initial water flush by directing the retentate valve to drain, and directing the entire permeate line to drain (not by the valve). Always run the pump at 1 LPM, i.e. if the retentate pressure is increased, the pump speed must also be increased to maintain 1 LPM. Rinse 4 L of USP water through both lines. Flush 2 L of 0.5 M sodium hydroxide with 250 ppm sodium hypochlorite made with USP water through both lines. Re-circulate 2 L of fresh solution through the system with the permeate line attached to the feed reservoir, and the retentate valve open to the reservoir for 60 minutes. Drain the solution through the bleed valve. Direct both lines to drain as in the initial flush. Fill the reservoir with USP water and drain 1 L through the bleed valve. Flush 8 L through both lines, and an additional 4 L through the permeate line with 5 psi of retentate pressure. 2 L of 0.4 M citric acid are then re-circulated through the system for 60 minutes. The acid solution is drained through the bleed valve, then the reservoir is filled with USP water and 1 L is drained through the bleed valve. 8 L of water is flushed through both the retentate and permeate lines, then and additional 8 L is flushed through the permeate at a cross flow of 1 LPM across the membrane with 5 psi of retentate pressure.

When both systems are cleaned and rinsed, they are assembled for storage (diagram above). 2 L of 0.1 M sodium hydroxide is poured into each feed vessel and pumped through the systems with the retentate and permeate valves open for recirculation, closed to waste, for 2 minutes. The vessels are then covered and status labeled as clean and stored in 0.1 M sodium hydroxide.

Process parameters have shown to be important in producing consistent material. The membranes used for the clarification are the CerCor ceramic 0.2 um pore size membrane, 1.5 sqft and the 30 kDa NMWCO Pall Filtron PES cassettes, 2 sq. ft. (2 cassettes). The temperature of the microfiltration system should be held between 26-29 C for optimum protein of interest clarity and flux. The microfiltration system should be run at a retentate flow rate of 14 LPM (42 cm/s) with a transmembrane pressure of 15 psig. The milk should be concentration down to 40-70% of the volume of the original pool (1.5-2.5×). The ultrafiltration portion of the system should be run at 1.6-2 LPM retentate flow rate with 20-30 psig of feed pressure. Permeate flow rate should be matched to that of the microfiltration system by adjusting the permeate pressures. The final bulk clarified concentrate should be one-quarter the volume of the original milk pool (4× concentration).

Recombinant Production

A growing number of recombinant proteins are being developed for therapeutic and diagnostic applications. However, many of these proteins may be difficult or expensive to produce in a functional form and/or in the required quantities using conventional methods. Conventional methods involve inserting the gene responsible for the production of a particular protein into host cells such as bacteria, yeast, or mammalian cells, e.g., COS or CHO cells, and then growing the cells in culture media. The cultured cells then synthesize the desired protein. Traditional bacteria or yeast systems may be unable to produce many complex proteins in a functional form. While mammalian cells can reproduce complex proteins, they are generally difficult and expensive to grow, and often produce only mg/L quantities of protein. In addition, non-secreted proteins are relatively difficult to purify from procaryotic or mammalian cells as they are not secreted into the culture medium.

In general, the transgenic technology features, a method of making and secreting a protein which is not normally secreted (a non-secreted protein). The method includes expressing the protein from a nucleic acid construct which includes: (a) a promoter, e.g., a mammary epithelial specific promoter, e.g., a milk protein promoter; (b) a signal sequence which can direct the secretion of a protein, e.g. a signal sequence from a milk specific protein; (c)optionally, a sequence which encodes a sufficient portion of the amino terminal coding region of a secreted protein, e.g., a protein secreted into milk, to allow secretion, e.g., in the milk of a transgenic mammal, of the non-secreted protein; and (d) a sequence which encodes a non-secreted protein, wherein elements (a), (b), optionally (c), and (d) are preferably operatively linked in the order recited.

In preferred embodiments: elements a, b, c (if present), and d are from the same gene; the elements a, b, c (if present), and d are from two or more genes.

In preferred embodiments the secretion is into the milk of a transgenic mammal.

In preferred embodiments: the signal sequence is the β-casein signal sequence; the promoter is the β-casein promoter sequence.

In preferred embodiments the non-secreted protein-coding sequence: is of human origin; codes for a truncated, nuclear, or a cytoplasmic polypeptide; codes for human serum albumin or other desired protein of interest.

The practice of the present invention will employ, unless otherwise indicated, conventional techniques of cell biology, cell culture, molecular biology, transgenic biology, microbiology, recombinant DNA, and immunology, which are within the skill of the art. Such techniques are described in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No: 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

Milk Specific Promoters

The transcriptional promoters useful in practicing the present invention are those promoters that are preferentially activated in mammary epithelial cells, including promoters that control the genes encoding milk proteins such as caseins, beta lactoglobulin (Clark et al., (1989) BIO/TECHNOLOGY 7: 487-492), whey acid protein (Gorton et al. (1987) Bio/Technology 5: 1183-1187), and lactalbumin (Soulier et al., (1992) FEBS LETTS. 297: 13). Casein promoters may be derived from the alpha, beta, gamma or kappa casein genes of any mammalian species; a preferred promoter is derived from the goat beta casein gene (DiTullio, (1992) Bio/Technology 10:74-77). The milk-specific protein promoter or the promoters that are specifically activated in mammary tissue may be derived from either cDNA or genomic sequences. Preferably, they are genomic in origin.

DNA sequence information is available for all of the mammary gland specific genes listed above, in at least one, and often several organisms. See, e.g., Richards et al., J. Biol. Chem. 256, 526-532 (1981) (α-lactalbumin rat); Campbell et al., Nucleic Acids Res. 12, 8685-8697 (1984) (rat WAP); Jones et al., J. Biol. Chem. 260, 7042-7050 (1985) (rat β-casein); Yu-Lee & Rosen, J. Biol. Chem. 258, 10794-10804 (1983) (rat γ-casein); Hall, Biochem. J. 242, 735-742 (1987) (ot-lactalbumin human); Stewart, Nucleic Acids Res. 12, 389 (1984) (bovine αs1 and κ casein cDNAs); Gorodetsky et al., Gene 66, 87-96 (1988) (bovine β casein); Alexander et al., Eur. J. Biochem. 178, 395-401 (1988) (bovine K casein); Brignon et al., FEBS Lett. 188, 48-55 (1977) (bovine αS2 casein); Jamieson et al., Gene 61, 85-90 (1987), Ivanov et al., Biol. Chem. Hoppe-Seyler 369, 425-429 (1988), Alexander et al., Nucleic Acids Res. 17, 6739 (1989) (bovine β lactoglobulin); Vilotte et al., Biochimie 69, 609-620 (1987) (bovine α-lactalbumin). The structure and function of the various milk protein genes are reviewed by Mercier & Vilotte, J. Dairy Sci. 76, 3079-3098 (1993) (incorporated by reference in its entirety for all purposes). To the extent that additional sequence data might be required, sequences flanking the regions already obtained could be readily cloned using the existing sequences as probes. Mammary-gland specific regulatory sequences from different organisms are likewise obtained by screening libraries from such organisms using known cognate nucleotide sequences, or antibodies to cognate proteins as probes.

Although the foregoing invention has been described in some detail by way of illustration and example for purposes of understanding, it will be apparent to those skilled in the art that certain changes and modifications may be practiced. Therefore, the description and examples should not be construed as limiting the scope of the invention, which is delineated by the appended claims.

Accordingly, it is to be understood that the embodiments of the invention herein providing for an improved method of high performance tangential flow filtration to generate a high yield of a molecule of interest from a given feedstream are merely illustrative of the application of the principles of the invention. It will be evident from the foregoing description that changes in the form, methods of use, and applications of the elements of the disclosed may be resorted to without departing from the spirit of the invention, or the scope of the appended claims.

### Prior Art Citations Incorporated by Reference

1. Aranha-Creado H, and Fennington G J Jr (1997, Cumulative Viral Titer Reduction Demonstrated By Sequential Challenge Of A Tangential Flow Membrane Filtration System And A Direct Flow Pleated Filter Cartridge, PDA J PHARM SCI TECHNOL 51(5):208-212.
2. Aravindan G R, et al., (1997), Identification, Isolation, And Characterization Of A 41-Kilodalton Protein From Rat Germ Cell-Conditioned Medium Exhibiting Concentration-Dependent Dual Biological Activities, ENDOCRINOLOGY 138(8):3259-68.
3. Christy C., et al., High Performance Tangential Flow Filtration: A Highly Selective Membrane Separation Process, DESALINATION, Vol. 144: 133-36 (2002).
4. Gabler et al., (1987), Principles of Tangential Flow Filtration: Applications to Biological Processing, in FILTRATION IN THE PHARMACEUTICAL INDUSTRY, pp. 453-490.
5. Ghosh R, et al., Parameter Scanning Ultrafiltration: Rapid Optimisation of Protein Separation, BIOTECHNOL BIOENG., 2003 March 20;81(6):673-82.
6. Kahn D W, et al., (2000), Purification of Plasmid DNA by Tangential Flow Filtration, BIOTECHNOL BIOENG. 69(1):101-106.
7. Kawahara H, et al., (1994), High-Density Culture of FM-3A Cells Using a Bioreactor With An External Tangential-Flow Filtration Device, CYTOTECHNOLOGY 14(1):61-66.
8. Koros, W. J. et al., (1996), Terminology for Membranes and Membrane Processes (IUPAC Recommendations 1996). PURE & APPL.CHEM. 68: 1479-89.
9. Millesime L, et al., Fractionation of Proteins with Modified Membranes, BIOSEPARATION, 1996 June;6(3):135-45.
10. Prado S M, et al., (1999), Development And Validation Study For The Chromatographic Purification Process For Tetanus Anatoxin On Sephacryl S-200 High Resolution, BOLL CHIM FARM. 138(7):364-368.
11. Porter, ed., HANDBOOK OF INDUSTRIAL MEMBRANE TECHNOLOGY, (Noyes Publications, Park Ridge, N.J., 1998) pp. 160-176.
12. Ramachandra-Rao, H. G. et al., Mechanisms of Flux Decline During Ultrafiltration of Dairy Products and Influence of pH on Flux Rates of Whey and Buttermilk, DESALINATION, vol. 144: 319-24 (2002).
13. Strauss P R (1995), Use Of Filtron Mini-Ultrasettetm Tangential Flow Device And Filtron Microseptm Centrifugal Concentrators In The Early Stages Of Purification Of DNA Polymerases, BIOTECHNIQUES 18(1):158-160.
14. Van Reis R., and Zydney A., Review, CURR OPIN BIOTECHNOL., 2001 April; 12(2):208-11.
15. Van Reis R., et al., High Performance Tangential Flow Filtration, BIOTECH. BIOENG., 56: 71-82, (1997).
16. Zeman, L. J. & Zydney, A. L. (1996), Microfiltration and Ultrafiltration, in PRINCIPLES AND APPLICATIONS. (Marcel Dekker ed.), New York.

### United States Patents and Patent Applications Incorpotated by Reference

1. Van Reis R. M., et al., U.S. Pat. No. 6,555,006, TANGENTIAL-FLOW FILTRATION SYSTEM.
2. Van Reis R. M., et al., U.S. Pat. No. 6,387,270, TANGENTIAL-FLOW FILTRATION SYSTEM.
3. Van Reis R. M., et al., U.S. Pat. No. 6,221,249, TANGENTIAL-FLOW FILTRATION SYSTEM.
4. Van Reis R. M., et al., U.S. Pat. No. 6,054,051, TANGENTIAL-FLOW FILTRATION SYSTEM.
5. Van Reis R. M., et al., U.S. Pat. No. 5,490,937, TANGENTIAL FLOW FILTRATION PROCESS AND APPARATUS.
6. Van Reis R. M., et al., U.S. Pat. No. 5,256,294, TANGENTIAL FLOW FILTRATION PROCESS AND APPARATUS.
7. Sandblom R. M. et al., U.S. Pat. No. 4,105,547, FILTERING PROCESS.
8. Jain M. et al., U.S. Pat. No. 4,351,710, FRACTIONATION OF PROTEIN MIXTURES.
9. Van Reis R. M., et al., United States Patent Application No.: 20020108907; TANGENTIAL-FLOW FILTRATION SYSTEM, filed Apr. 12, 2002.

1. A method for separating a protein of interest from a feedstream, comprising:
(a) filtering said feedstream by a high performance tangential-flow filtration process that separates said molecular species of interest from said feedstream on the basis of pore size and charge of said protein of interest, while maintaining flux at a level ranging from about 5 to 100% of transition point flux in the pressure-dependent region of the flux versus TMP curve, wherein transmembrane pressure is held substantially constant along the membrane at a level no greater than the transmembrane pressure at the transition point of the filtration, whereby said protein of interest is selectively separated from said feedstream such that said protein of interest retains its biological activity;
(b) filtering said feedstream by an ultrafiltration process; and,
wherein said filtration is occurring above the transition point flux of said protein of interest;
wherein said molecular species has a molecular weight of between 1 and 1000 kDa.

2. The method of claim 1, further comprising fractionating said feedstream.

3. The method of claim 1, further comprising clarifying said feedstream.

4. The method of claim 1, further comprising diafiltering said feedstream.

5. The method of claim 1, further comprising increasing transmembrane pressure and decreasing flux for the first half of the filtration.

6. The method of claim 5, further comprising decreasing transmembrane pressure for the second half of the process.

7. The process of claim 1 wherein said feedstream is concentrated before filtration.

8. The process of claim 1 wherein said protein of interest is less than ten times larger or smaller in molecular weight than a second protein of interest in said feedstream.

9. The process of claim 1 wherein the protein of interest is more than ten times larger or smaller in molecular weight than a second protein of interest of the mixture but have the same charge or isoelectric point.

10. The method of claim 1, further comprising concentrating said feedstream.

11. The method of claim 1, wherein all filtration stages are ultrafiltrations.

12. The method of claim 1, wherein said feedstream is milk

13. The method of claim 1, wherein said feedstream is a cell lysate solution.

14. The method of claim 1, wherein said protein is a biopharmaceutical.

15. The method of claim 12, wherein the condition of said milk is selected from one of the following states:
a) raw;
b) diluted;
c) treated with a buffer solution;
d) chemically treated; and
e) partially evaporated.

16. The method of claim 2, wherein said fractionation step utilizes ceramic filtration membranes.

17. The method of claim 3, wherein said clarification step utilizes ceramic filtration membranes.

18. The method of claim 2, wherein said fractionation step utilizes polymeric filtration membranes with a defined isoelectric profile.

19. The method of claim 3, wherein said clarification step utilizes polymeric filtration membranes.

20. The method of claim 2, wherein said fractionation step utilizes cellulose filtration membranes.

21. The method of claim 3, wherein said clarification step utilizes cellulose filtration membranes.

22. The method of claim 2, further comprising optimizing systematic parameters.

23. The method of claim 22, wherein said systematic parameters include temperature, feedstream flow velocity, transmembrane pressure, feedstream concentration and diafiltration volume.

24. The method of claim 3, further comprising optimizing systematic parameters.

25. The method of claim 24, wherein said systematic parameters include temperature, feedstream flow velocity, transmembrane pressure, feedstream concentration and diafiltration volume.

26. The method of claim 1 wherein said molecular species of interest are biological entities selected from the group consisting of proteins, immunoglobulins, polypeptides, peptides, glycoproteins, RNA and DNA.

27. The method of claim 23, wherein the optimal temperature range is from 15° C. to 50° C.

28. The method of claim 23, wherein the optimal temperature range is from 20° C. to 35° C.

29. The method of claim 23, wherein the optimal temperature range is from 25° C. to 29° C.

30. The method of claim 25, wherein the optimal temperature range is from 15° C. to 50° C.

31. The method of claim 25, wherein the optimal temperature range is from 20° C. to 35° C.

32. The method of claim 25, wherein the optimal temperature range is from 25° C. to 29° C.

33. The method of claim 23, wherein the feedstream flow velocity is from 10 cm/sec to 100 cm/sec.

34. The method of claim 23, wherein the feedstream flow velocity is from 20 cm/sec to 60 cm/sec.

35. The method of claim 23, wherein the feedstream flow velocity is from 25 cm/sec to 45 cm/sec.

36. The method of claim 25, wherein the feedstream flow velocity is from 10 cm/sec to 100 cm/sec.

37. The method of claim 25, wherein the feedstream flow velocity is from 20 cm/sec to 60 cm/sec.

38. The method of claim 25, wherein the feedstream flow velocity is from 25 cm/sec to 45 cm/sec.

39. The method of claim 23, wherein the transmembrane pressure ranges from 2 psi to 40 psi.

40. The method of claim 23, wherein the transmembrane pressure ranges from 5 psi to 30 psi.

41. The method of claim 23, wherein the transmembrane pressure ranges from 10 psi to 20 psi.

42. The method of claim 25, wherein the transmembrane pressure ranges from 2 psi to 40 psi.

43. The method of claim 25, wherein the transmembrane pressure ranges from 5 psi to 30 psi.

44. The method of claim 25, wherein the transmembrane pressure ranges from 10 psi to 20 psi.

45. The method of claim 23, wherein the feedstream concentration is from 0.25× to 4× natural milk.

46. The method of claim 23, wherein the feedstream concentration is from 0.5× to 3× natural milk.

47. The method of claim 23, wherein the feedstream concentration is from 1.0× to 2× natural milk.

48. The method of claim 25, wherein the feedstream concentration is from 0.25× to 4× natural milk.

49. The method of claim 25, wherein the feedstream concentration is from 0.5× to 3× natural milk.

50. The method of claim 25, wherein the feedstream concentration is from 1.0× to 2× natural milk.

51. The method of claim 23, wherein the diafiltration volume range is from 1× to 20× the volume of concentrated MF retentate.

52. The method of claim 23, wherein the diafiltration volume range is from 3× to 15× the volume of concentrated MF retentate.

53. The method of claim 23, wherein the diafiltration volume range is from 5× to 10× the volume of concentrated MF retentate.

54. The method of claim 25, wherein the diafiltration volume range is from 1× to 20× the volume of concentrated MF retentate.

55. The method of claim 25, wherein the diafiltration volume range is from 3× to 15× the volume of concentrated MF retentate.

56. The method of claim 25, wherein the diafiltration volume range is from 5× to 10× X the volume of concentrated MF retentate.

57. The method of claim 2, wherein ultrafiltration membranes are used for all filtering steps.

58. The method of claim 7, wherein ultrafiltration membranes are used for all filtering steps.

59. The method of claim 12, wherein said milk is treated with a solution selected from the group consisting of:
a) water;
b) a buffered aqueous salt solution;
c) chelating agent;
d) acid solution; and
e) alkali solution.

60. The method of claim 4, wherein said diafiltration utilizes ultrafiltration permeate.

61. The method of claim 4, wherein said diafiltration utilizes water.

62. The method of claim 4, wherein said diafiltration utilizes a buffered salt solution.

63. The method of claim 1, wherein the membranes used are cleaned with solutions of a temperature greater than 20° C.

64. The method of claim 1, wherein the membranes used are cleaned with solutions ranging in temperature from 20° C. to 70° C.

65. The method of claim 1, wherein the membranes used are cleaned with solutions ranging in temperature from 40° C. to 60° C.

66. The method of claim 1, wherein the membranes used are cleaned with an acid solution.

67. The method of claim 1, wherein the membranes used are cleaned with an alkali solution.

68. The method of claim 1, wherein the membranes used are cleaned with a hypochlorite solution.

69. The method of claim 66, 67 or 68, further comprising a water rinse following the use of the selected solution.

70. The method of claim 1, wherein the membranes used are sanitized prior to use with a hydroxide solution.

71. The method of claim 1, wherein the membranes used are sanitized prior to use with an alcohol solution.

72. The method of claim 1, wherein the membranes used are sanitized prior to use with a hypochlorite solution.

73. The method of claim 1, wherein the membranes used are cleaned for a period of from 20 minutes to 45 minutes.

74. The method of claim 1, further comprising filtering the filtrate from the filtration in a second tangential-flow filtration stage through a membrane having a smaller pore size than the membrane used in the first filtration stage, and recycling the filtrate of this second filtration stages back to the first filtration stage, whereby the process is repeated.

75. A method for separating a protein of interest from a feedstream, comprising:
(a) filtering said feedstream by a high performance tangential-flow filtration process that separates said molecular species of interest from said feedstream on the basis of pore size and charge of said protein of interest, while maintaining flux at a level ranging from about 5 to 100% of transition point flux in the pressure-dependent region of the flux versus TMP curve, wherein transmembrane pressure is held substantially constant along the membrane at a level no greater than the transmembrane pressure at the transition point of the filtration, whereby said protein of interest is selectively separated from said feedstream such that said protein of interest retains its biological activity;
(b) filtering said feedstream by a microfiltration process; and,
(c) increasing transmembrane pressure and decreasing flux for the first half of the filtration;
(d) decreasing therafter increasing or maintaining flux as the filtration progresses.
wherein said filtration is occurring above the transition point flux of said protein of interest;
wherein said molecular species has a molecular weight of between 1 and 1000 kDa.

76. The methods of claim 1 or 75 wherein said protein of interest is recombinant human alphafetoprotein.

77. The methods of claim 1 or 75 wherein said protein of interest is recombinant human albumin.

78. The methods of claim 1 or 75 wherein said protein of interest is sourced from the milk of a transgenic mammal.
